(19) United States
(12) Patent Application Publication
(10) Pub. No.: US 2014/0121355 A1
(43) Pub. Date: May 1, 2014

(54) Drug Screening Target For Alzheimer's Disease and Method of Screening Potential Drugs

(71) Applicants: JOSEPH AUDIE, Meriden, CT (US); Sergei Yarosiavovitch Ponomarev, Worcester, MA (US)

(72) Inventors: JOSEPH AUDIE, Meriden, CT (US); Sergei Yarosiavovitch Ponomarev, Worcester, MA (US)

(21) Appl. No.: 14/093,631

(22) Filed: Dec. 2, 2013

Related U.S. Application Data
(62) Division of application No. 13/423,239, filed on Mar. 18, 2012, now abandoned.
(60) Provisional application No. 61/453,703, filed on Mar. 17, 2011.

(51) Int. Cl. C07K 14/705

(52) U.S. Cl. CPC C07K 14/705
USPC 530/324

(57) ABSTRACT

Drug screening targets and method of screening for potential drugs for treatment or amelioration of Alzheimer's Disease are provided.

### CROSS-REFERENCE TO RELATED APPLICATION

This application is a divisional of U.S. patent application Ser. No. 13/423,239, filed Mar. 18, 2012, which claims the benefit of U.S. provisional patent application 61/453,703, filed Mar. 17, 2011, each of which is incorporated herein by reference in its entirety.

### FIELD OF THE INVENTION

This invention relates to the field of drug targets relevant to the etiology, study, and treatment of Alzheimer's disease and to methods for screening chemical compounds to determine their potential utility for treatment or amelioration of Alzheimer's Disease.

### BACKGROUND

Alzheimer's disease (AD) is a degenerative affliction of the nervous system that negatively impacts a person's memory, cognitive functions, and ability to perform the normal activities of daily living. The disease also causes behavioral problems with which the families of those with the disease must cope. Typically, AD reduces the lifespan of an individual by increasing an afflicted person's risk of succumbing to secondary infections and illnesses. AD will become increasingly common during the next three decades as the American population—in particular, the “baby-boom” generation—ages. It is estimated that by 2035, when the average age of the baby boom generation is 85, up to 50% of Americans will have developed AD. Alzheimer's disease is associated with the accumulation of beta-amyloid plaques in the brain that lead to the eventual destruction of brain cells. The primary cause of AD may be flaws in the metabolic processes governing production, accumulation, or disposal of the beta-amyloid protein fragments. Therefore, treatments for AD often have focused on dissolving beta-amyloid or preventing the aggregation of the beta-amyloid fragments into plaque formations.

Recently, a novel molecular mechanism to account for axonal pruning and neuronal cell death during physiological development has been described. It is further hypothesized that the new mechanism has implications for the pathophysiology of AD. According to their proposed developmental model, tropic factor deprivation results in amyloid precursor protein (APP) proteolysis, culminating in the release of an N-terminal APP fragment (NAPP) into the extracellular milieu. NAPP then serves as a ligand for death cell receptor six (DR6), a member of the tumor necrosis factor receptor (TNFR21) family. Binding to the DR6 ectodomain results in the subsequent downstream activation of caspase-3 and caspase-6, respectively, resulting in accelerated neuronal apoptosis, neuronal degeneration, axonal degeneration, and the physiological sculpting of nerve connections in the developing brain. It is proposed that this physiological pathway could be hijacked in the adult brain, resulting in AD. The DR6-GFD NAPP protein-protein interaction, then, is a key event in the pathway described, and possibly in the progression of AD.

### SUMMARY OF THE INVENTION

The present invention provides compositions and methods for discovering molecules that have the potential to interfere with the DR6-GFD NAPP interaction, thus treating or ameliorating AD.

In one aspect, the present invention is directed towards a polypeptide whose amino acid residues have about 30% homology to residues 38-123 of the growth factor-like domain (GFD) of the N-terminal APP fragment (NAPP). The polypeptide adopts a specific conformation in vivo characterized by having seven beta strands. In addition, the residues 66-81 of the polypeptide adopt a lone alpha-helix motif. Finally, residue 62 of the polypeptide is Cysteine, residue 71 is Glutamine, residue 74 is Glutamine, residue 82 is Isoleucine, residue 103 is Lysine, and residue 123 is Valine. In other aspects, the invention is directed to such a polypeptide whose amino acid residues have about 40%, about 50%, about 75%, about 90%, or 100% homology to residues 38-123 of the NAPP.

In another aspect, the present invention is directed towards a polypeptide whose amino acid residues have about 30% homology to residues 96-167 of Death Cell Receptor 6 (DR6), including a first Cysteine Rich Domain (CRD) with at least 30% homology to amino acid residues 96 to 131 of DR6 and a second Cysteine Rich Domain (CRD) with at least 30% homology to amino acid residues 133 to 167 of DR6. The polypeptide adopts a specific conformation in vivo characterized by having twelve beta strands. In addition, residue 98 of the polypeptide is Arginine, residue 104 is Glutamate, residue 131 is Cysteine, residue 132 is Threonine, residue 139 is Glutamine, residue 163 is Threonine, and residue 167 is Arginine. In other aspects, the invention is directed to such a polypeptide whose amino acid residues have about 40%, about 50%, about 60%, about 75%, about 85%, or 100% homology to residues 96-167 of the DR6.

In another aspect, the present invention is directed toward methods for screening chemical compounds to determine their potential to modulate or bind to DR6 to prevent or inhibit its binding to GFD NAPP or to bind to GFD NAPP to prevent or inhibit its binding to DR6. In still another aspect, the present invention is directed toward methods for screening chemical compounds to determine their potential to treat, ameliorate or retard the onset of AD.

### BRIEF DESCRIPTION OF THE FIGURES

FIG. 1 is a ribbon representation of the GFD NAPP crystal structure with key residues highlighted.

FIG. 2 is a ribbon representation of the refined DR6 ectodomain homology model.

FIG. 3 is a ribbon representation for the best DR6-GFD NAPP model.

FIG. 4 shows the sequence alignment and secondary structure of the growth factor-like domain of human N-terminal APP (GFD NAPP, SEQ ID NO: 1) and APLP2 (SEQ ID NO: 3).

FIG. 5 shows ribbon representations of GFD NAPP along with 22C11 interface residues (a), ClusPro predicted interface residues (b), and PPI-Pred predicted interface residues (c).

FIG. 6 shows sequence alignment and secondary structure of the human DR6 ectodomain (SEQ ID NO: 2) and its homolog, p75 (SEQ ID NO: 4).

FIG. 7 shows ribbon representations of the DR6 ectodomain homology model (a), ClusPro predicted (b), and PPI-Pred predicted interface residues (c).

FIG. 8 shows the final DR6-GFD NAPP-docked structure (a), final structure of the DR6-NGF NAPP complex (b), DR6-GFD NAPP hydrogen bonds and salt bridges (c), and rigid body association of the DR6-NGF NAPP complex (d).

FIG. 9 shows a comparison of the p75-NGF crystal structure with the best re-docked p75-NGF model.

FIG. 10 shows the observed and predicted interface residues for the p75 receptor derived from the x-ray structures, with interface residues (a), ClusPro predicted interface residues (b), and PPI-Pred predicted interface residues (c).

FIG. 11 shows the NGF ligand, with interface residues (a), ClusPro predicted interface residues (b), and PPI-Pred predicted interface residues (c).

FIG. 12 shows the model structure of p75-GFD NAPP.

### DETAILED DESCRIPTION OF THE INVENTION

One aim of the present invention is to construct a theoretical model of the DR6-GFD NAPP interaction that will lead to the discovery of compounds useful for the treatment or amelioration of AD. A DR6-GFD NAPP interaction model is constructed using homology modeling, rigid-body docking and free energy scoring. Calculations and model predictions are compared, to the extent permitted by the available data, with experimental results and independently generated theoretical results. The final model is analyzed to indicate the physical basis of DR6-GFD NAPP recognition, especially within the context of known TNFR interactions.

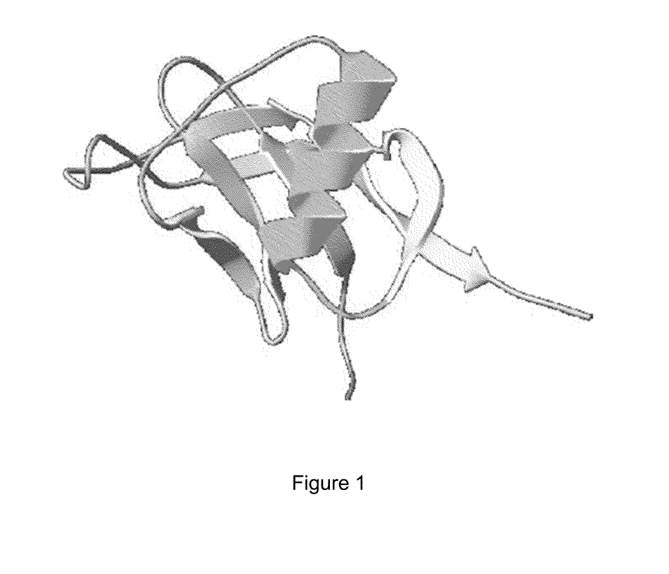

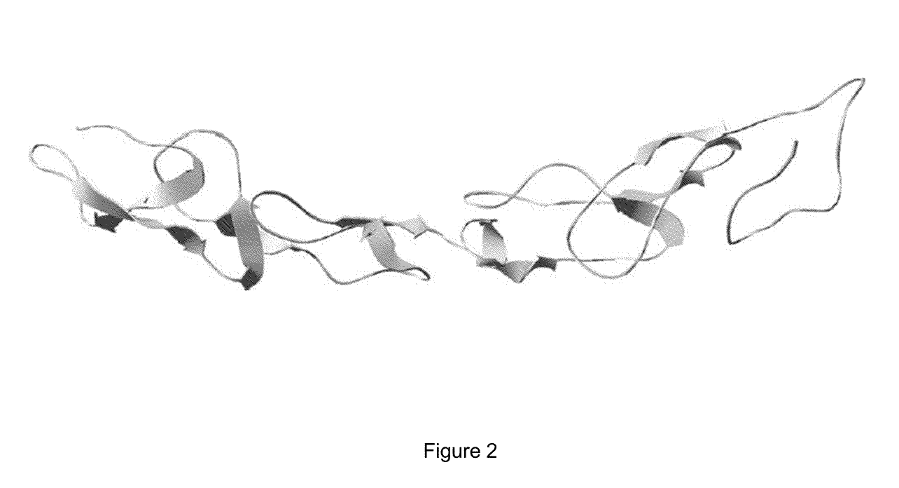

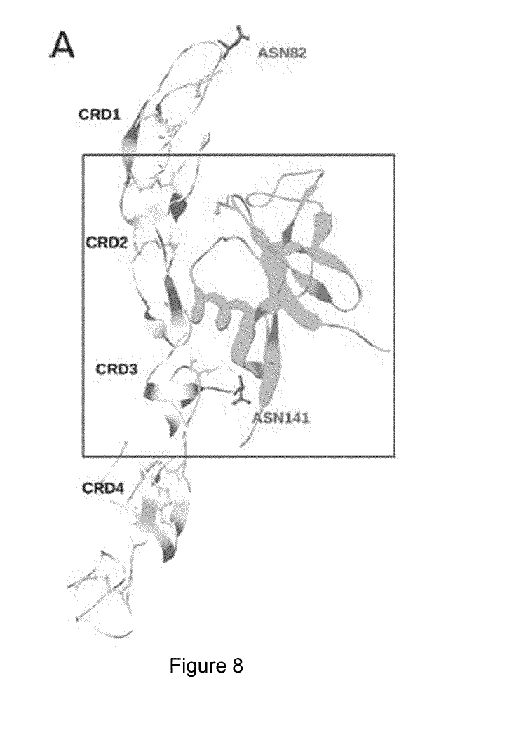

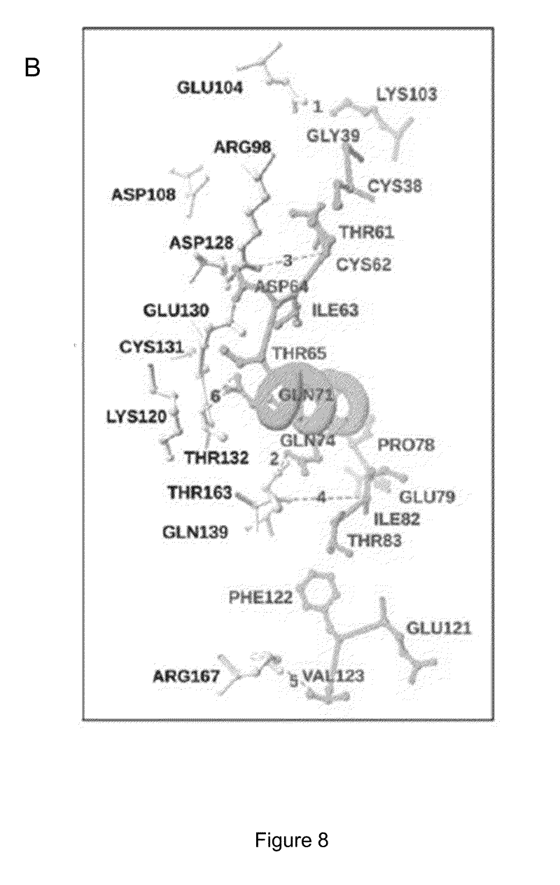

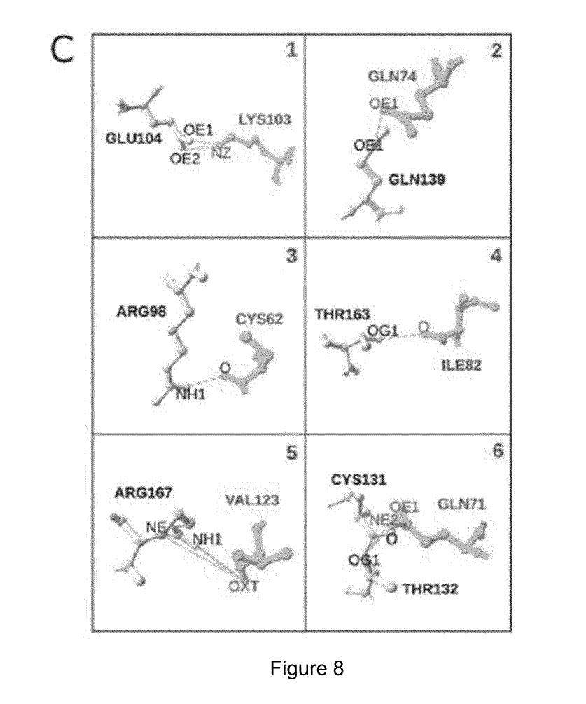

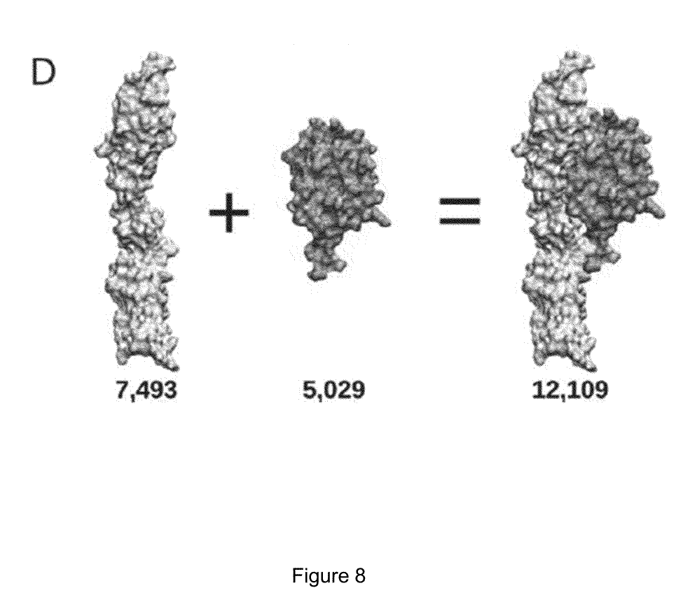

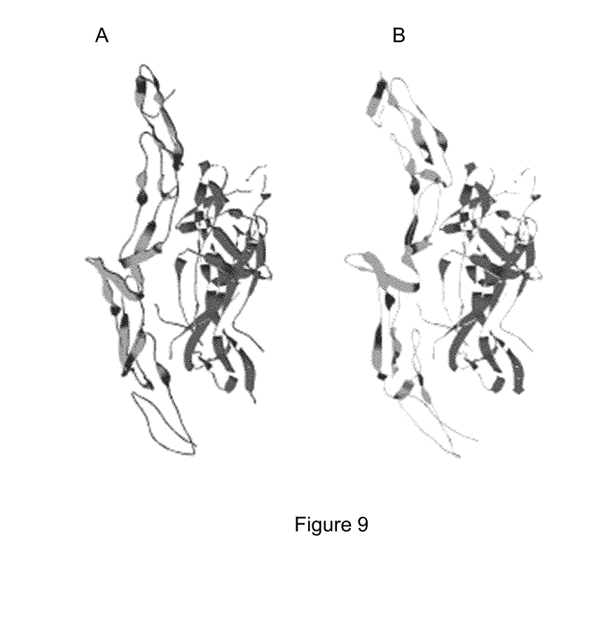

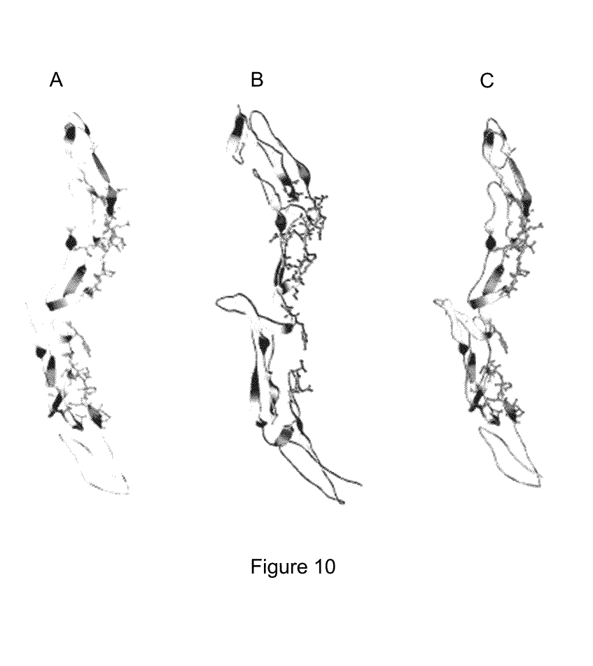

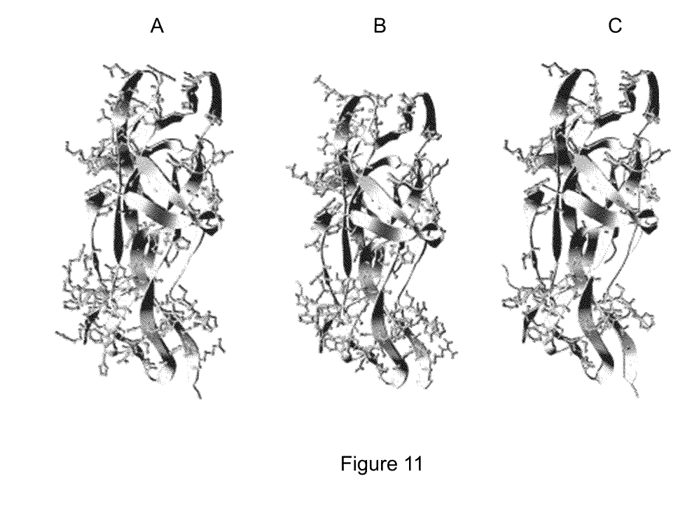

The crystal structure of residues 28-123 of GFD NAPP has been solved at 1.8 Å resolution. The structure is available in the Protein Data Bank (PDB) (http://www.pdb.org/) with PDB identifier “1 mwp.” The high quality of the GFD NAPP crystal structure is verified using standard tools. Comparison with a second, lower resolution but bound NAPP dimer structure (PDB identifier “3ktm”) indicates that the 1 mwp GFD NAPP structure represents a realistic binding competent conformation. As such, the 1 mwp structure is used in the present study and docked to a homology model of the DR6 ectodomain. The protein models and pictures, with an exception of FIG. 8b which is done in Visual Molecular Dynamics (VMD) (http://www.ks.uiuc.edu/Research/vmd/), are prepared using Swiss PDB Viewer (http://spdbv.vital-it.ch/). FIG. 1 provides a ribbon representation of the GFD NAPP crystal structure (28-123). GFD NAPP is a high resolution high quality crystal structure that exhibits a globular fold. Several key residues (66-81) comprise a lone alpha-helix-loop motif.

In order to construct a binding competent theoretical model of the DR6 ectodomain, a homology modeling program and/or server may be used. An exemplary embodiment of such a modeling program and/or server (which is discussed for consistency and clarity herein is the I-TASSER homology modeling server (http://zhang.bioinformatics.ku.edu/I-TASSER/). Other representative examples which may be used include: 3d-jigsaw, Selvita Protein Modeling Platform, ROBETTA, Rosetta, CABS, Swiftmodel, LOOPP, RAPTOR, and SPARKSx.

Towards that end, DR6 residues 67-211 are submitted to the I-TASSER server. The I-TASSER server builds homology models through an exhaustive process that involves automatic template selection, fragment reassembly of aligned regions, ab initio modeling of unaligned regions, clustering, energy evaluation and the optimization of a model's hydrogen bonding network. Ultimately, the top ranked I-TASSER DR6 model is selected based on the template supplied by the bound crystal coordinates of the neurotrophin receptor p75 in complex with the neurotrophin (NGF) ligand (PDB code: 1sg1, chain X). For further analysis and eventual docking, it is necessary to employ the p75 template (identified by the I-TASSER server due to its sequence homology with DR6) since the structure of the DR6 ectodomain is unavailable. The p75 ectodomain shares good sequence identity with the DR6 sequence. Like DR6, p75 is a transmembrane protein, is a member of the TNFR family (TNFR16), plays a role in apoptosis and in AD, and is known to bind NAPP.

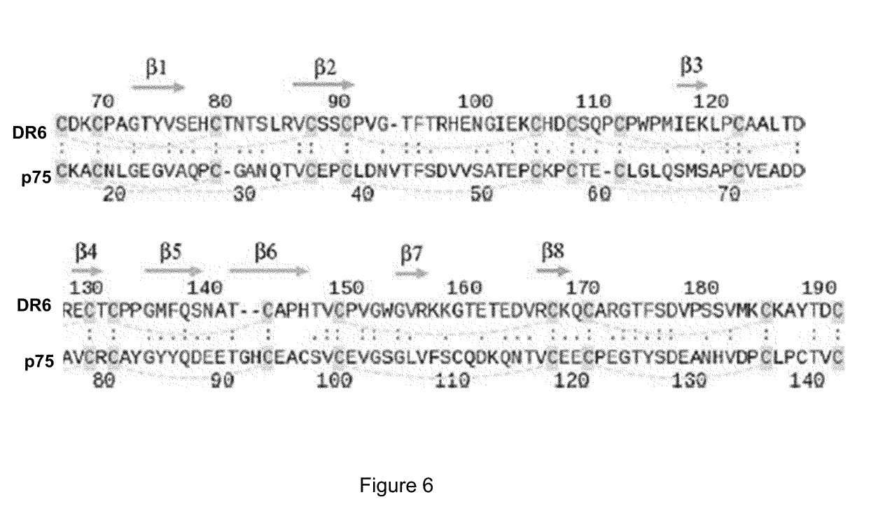

Thus, like the DR6 ectodomain the p75 ectodomain is stabilized by numerous disulfide bonds and is organized into several cysteine-rich domains (CRD) that is seen to play a role in binding. The p75-NGF interaction has also been the subject of previous modeling and docking studies. Thus, using p75 as a template structure, we are able to construct a high quality and binding competent homology model of the DR6 ectodomain. The sequence alignment and secondary structure of the human DR6 ectodomain and p75 are depicted in FIG. 6.

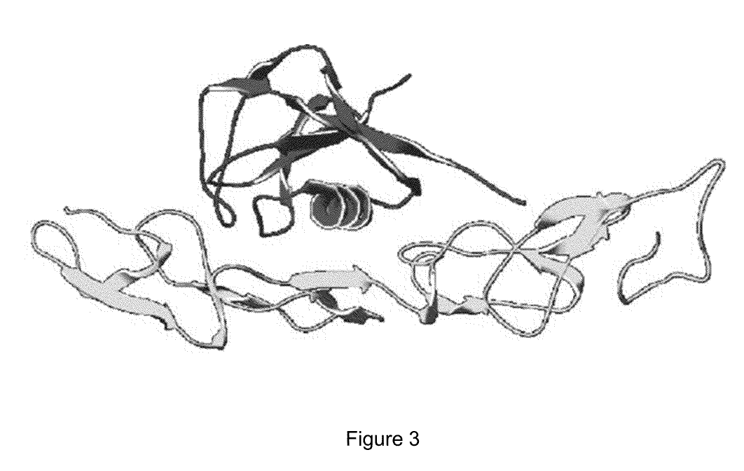

FIG. 2 shows a ribbon representation of our energy optimized and refined DR6 ectodomain homology model. Table 1 summarizes an evaluation of the model by using a variety of computational tools. The ectodomain of DR6 comprises residues 67-211. The model is constructed using the bound coordinates of the p75 receptor and represents a binding competent conformation. The DR6 ectodomain model takes on a more extended shape and exhibits beta secondary structure interconnected through less well defined structural elements. The DR6 structure forms a structural depression or basket region that seems well suited to accommodate a globular protein such as GFD NAPP.

TABLE 1

| | DR6 | Minimized | |
|---|---|---|---|
| | I-TASSER | DR6 I-TASSER | p75 template |
| Model Quality | model | model | structure (1sg1) |
| I-TASSER C-score* | 1.31 | N/A | N/A |
| I-TASSER TM-score* | 0.9 | N/A | N/A |
| I-TASSER RMSD* | 2.2 | N/A | N/A |
| ProSA Z-Score** | −3.87 | −4.33 | −4.33 |
| QMEAN Score*** | 0.530 | 0.454 | 0.441 |
| DFIRE Energy**** | −109.80 | −124.85 | −126.03 |
| Minimization | N/A | −4564.22 | N/A |
| Energy***** | | | |

*I-TASSEER server http://zhang.bioinformatics.ku.edu/I-TASSER/
**ProSA Server https://prosa.services.came.sbg.ac.at/prosa.php
***Qmean Server swissmodel.expasy.org/qmean/
****SWISS-MODEL DFIRE http://swissmodel.expasy.org/workspace/
*****TINKER GB/SA AMBER99 minimization (kcal/mol) http://dasher.wustl.edu/ffe/

Homology model construction is typically followed by visual and quantitative model evaluation. Importantly, the I-TASSER server automatically calculates and outputs various quality scores to assist end-users in model evaluation and selection. In particular, I-TASSER calculates an overall target quality score and a predicted target TM score and RMSD score. The quality of our DR6 ectodomain homology model is further assessed according to a Ramachandran map analysis and through the use of three independent server-based methods: ProSA, Qmean and DFIRE.

The ProSA server is available at: https://prosa.services.came.sbg.ac.at/prosa.php. Qmean and DFIRE are accessed through the SWISS-MODEL server (http://swissmodel.expasy.org/). All three servers use disparate methods to calculate quantitative scores that can be used to asses model quality and guide model selection.

Refining the Structure of the I-TASSER Ectodomain DR6 Model.

We use energy minimization, along with the Amber99 force field and the GB/SA implicit solvent model, to refine our DR6 ectodomain model. A termination criterion of 0.5 kcal/mol is applied and convergence is achieved. All calculations are carried out using the TINKER molecular modeling package (http://dasher.wustl.edu/tinker/).

The energy minimized I-TASSER DR6 ectodomain homology model is then used in the rigid-body protein-protein docking study.

One goal is to generate a reasonably accurate model of the interaction between the DR6 ectodomain and GFD NAPP. To achieve this goal we use the refined I-TASSER DR6 ectodomain model along with the GFD NAPP crystal structure as inputs to the ClusPro Docking server, version 1.0 (http://nrc.bu.edu/cluster/cluspro_v1.cgi).

By default, the ClusPro server docks receptor (DR6) and ligand (GFD NAPP) structures using version 1.0 of the DOT rigid-body docking algorithm (http://www.sdsc.edu/CCMS/DOT/). The top 20,000 complexes generated by DOT are then filtered according to electrostatic and desolvation energies and the top 2,000 complexes are retained for further processing. The retained 2,000 conformations are then clustered according to interface RMSD and the top 10 docked models, following a short Charmm19 energy minimization, are made available for download. The top 10 ClusPro models capture most of the important rigid-body binding geometries and provide excellent starting structures for further refinement and analysis. The ClusPro docking methodology is validated using the 1sg1 crystal structure.

Ultimately, the top 10 ClusPro models are narrowed down to a single physically realistic docked configuration. To accomplish this, the binding affinities of the top 10 ClusPro conformations are estimated in a hierarchical fashion. First, all 10 complexes are relaxed and optimized through rigid-body energy minimization using the Charmm19 force field. Next, a pseudo-binding affinity (ΔG_{bind,MM-GB/SA}) is calculated for all 10 models using the Charmm19 molecular-mechanics force field and GB/SA implicit solvent model (MM-GB/SA). All calculations are made using TINKER and default settings. Finally, ClusPro generated complexes with negative pseudo-binding affinities (ΔG_{MM-GB/SA}<0) are scored using a recently described empirical free energy function that is available through CMDBioscience (http://www.cmdbioscience.com/).

The present invention's approach to protein-protein and protein-peptide binding free energy prediction (ΔG_{bind,empirical}) involves the use of a novel, fast, physics-based, empirical free energy function. The function is a six-term, regression-weighted expression and is given by:

ΔG_{bind,empirical}=0.79ΔX_{+/-}+0.075ΔX_{c/s}−0.65X_{sb}−0.86X_{hb}−0.00089X_{gap}−0.089ΔXₜₒᵣ−0.33 (1)

The first two terms refer to binding-induced changes in the total number of solvent-exposed charged atoms (N-terminal nitrogen atoms, Arg and Lys side chain nitrogen atoms; O-terminal oxygen atoms, Asp and Glu side-chain carboxyl oxygen atoms; by default, His is treated as uncharged) and hydrophobic atoms (C and S atoms), respectively. The third and fourth terms refer to the total number of hydrogen bonds and the net number (difference between favorable and unfavorable charge-charge contacts) of short-range (≦4 Å) charge-charge or salt bridge interactions across the protein-protein interface. The contributions of these pairwise interface hydrogen bonding and salt bridge interactions are penalized according to the degree of solvent exposure, such that if the average solvent exposure is greater than some experimentally derived threshold value, energetic penalties are added to Eq. (1). The final three descriptors, in order, refer to the interface gap or void volume, the change in the number of solvent exposed side-chain torsions or the total number of side chain torsions buried at the interface, and a constant contribution. Changes in the number of solvent exposed main-chain torsions can also be counted for peptide ligands. Theoretical and empirical considerations indicate that Eq. (1) will produce accurate absolute binding affinity predictions only for receptor-ligand reactions that approximate rigid-body association. Default values are used for each descriptor and all other important quantities. The model complex with the lowest empirical free energy score (most negative predicted absolute binding affinity) is ultimately selected as the best DR6-GFP NAPP structural interaction model. The refinement and scoring procedure is validated using the 1sg1 crystal structure.

A single, physically realistic, DR6-GFD NAPP predicted complex structure is identified. Importantly, the modeling workflow incorporates extensive a priori testing to ensure the physical reasonableness and accuracy of the model. FIG. 3 provides a ribbon representation of the identified DR6-GFD NAPP interaction model (model 1). The model indicates an important recognition role for the GFD NAPP alpha-helix-loop motif (residues 66-81). The model also indicates that the GFD NAPP alpha helix (66-76) rests in or lines the previously mentioned DR6 structural depression or basket.

A priori considerations demonstrated that model 1 is of probable accuracy. The model is then further tested model 1 a posteriori. Testing is divided into two categories: (1) biophysical testing and (2) theoretical testing of the model. The biophysical model testing phase involves binding affinity comparisons, the analysis of GFD NAPP and DR6 sequence alignments, and a comparative analysis with the available anti-GFD NAPP 22C11 antibody data. The theoretical model testing phase involves comparisons between data derived from our DR6-GFD NAPP model and independently generated computational data.

In addition to binding NAPP, the DR6 ectodomain binds the N-terminus of APLP2 and with similar affinity. By inferring that APLP2 adopts a similar binding configuration to DR6 as does GFD NAPP, we further infer that a sequence alignment between APLP2 and the interface residues of GFD NAPP will reveal significant conservation. Thus, the predicted interface residues of GFD NAPP are compared, derived from our DR6-GFD NAPP model, with the aligned residue positions of APLP2. The empirically calculated binding affinity of the best docked model (−11.1 kcal/mol) is in excellent agreement with the experimentally estimated binding free energy (−11.5 kcal/mol). This alignment reveals that the GFD NAPP interface residues align almost perfectly with the APLP2 residues that probably mediate binding to DR6. This provides indirect evidence that the residue-level contribution of GFD NAPP to DR6 binding is captured the model.

It has also been shown that the NAPP antibody 22C11 interferes with DR6-NAPP binding. Importantly, it has also been shown that the binding epitope recognized by the 22C11 antibody spans NAPP residues 66-81. This represents a stretch of residues that are localized around the lone helix (66-76) of GFD NAPP. On the inference that 22C11 blocks DR6 binding to NAPP by binding to the same GFD NAPP surface that mediates DR6-GFD NAPP interaction, we compared the GFD NAPP interface residues derived from our model with the GFD NAPP epitope that is known to bind 22011. Once again, that good agreement between the two indicates verification of the model. Using a 4.5 Å cutoff criterion, the GFD NAPP residues that line the DR6-GFD NAPP interface of our model include residues 67, 68, 70, 71, 74, 78, and 79. Thus, there exists excellent agreement between the experimentally determined 22011 epitope and the interface residues of our model. Thus, the modeled GFD NAPP contribution to DR6 binding enjoys further verification and, moreover, focuses attention on the specific role played by helix residues 66-76 in DR6-NAPP recognition.

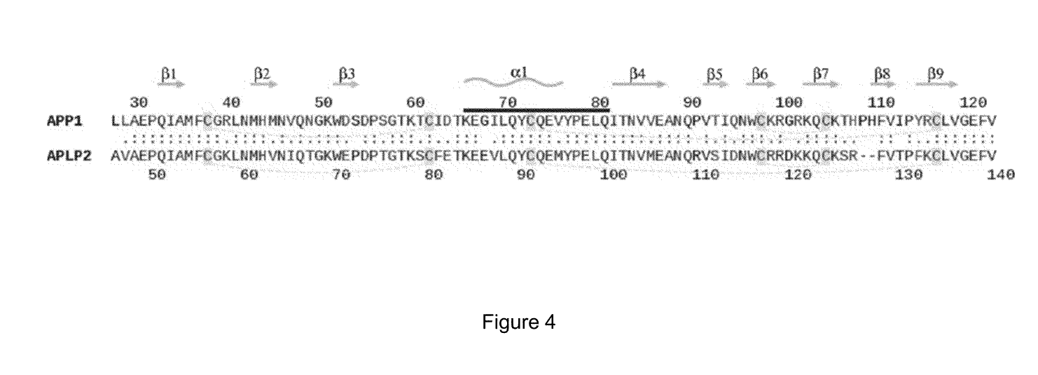

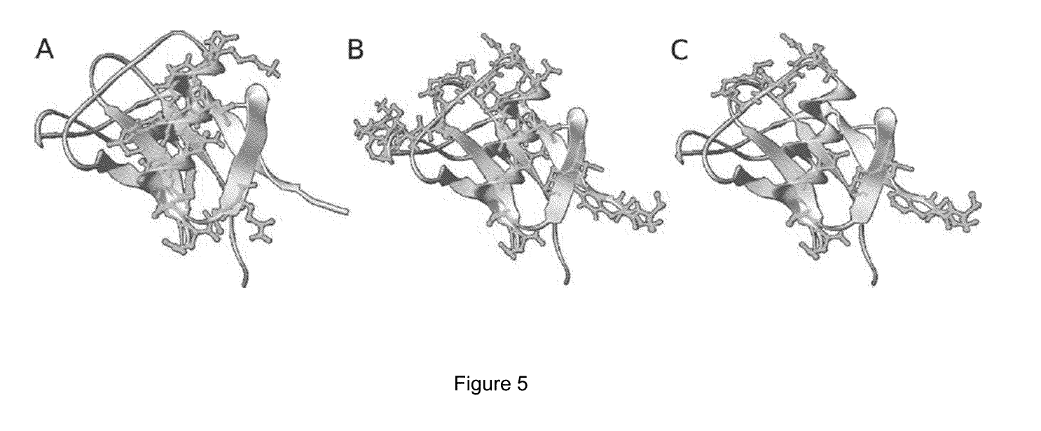

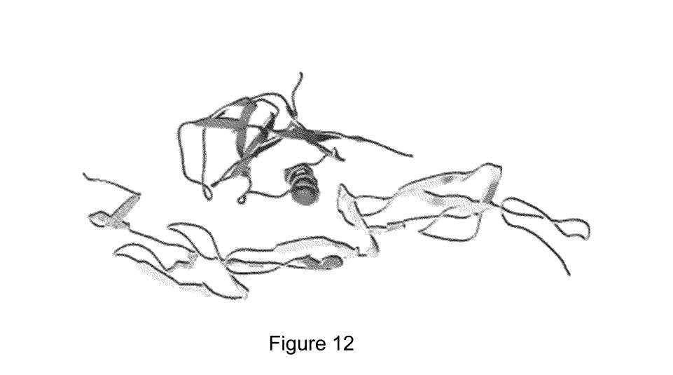

FIG. 4 shows the sequence alignment and secondary structure of the growth factor-like domain of human N-terminal APP (GFD NAPP) and APLP2; secondary structural information is also presented. The anti-GFD NAPP (22011) antibody binding epitope is indicated by a solid black line. FIG. 5a shows structural models that depict interface residues derived from the 22011 antibody binding experiments. Structural supposition is used to model the p75-NAPP interaction (FIG. 12) and compare the theoretical estimate of binding affinity with the experimental value (Table 1b), thus validating the homology server's selection of p75 as a template for secondary structure prediction of DR6.

TABLE 1b

| | Predicted ΔG_{bind,empirical} | Experimental ΔG_{bind,exp} |
|---|---|---|
| Complex | (kcal/mol) | (kcal/mol) |
| P75-GFD NAPP | −7.6 | −9.0 |

The theoretical testing involved a comparison between the interface residues derived from our docked model with predicted binding site or interface residues for GFD NAPP and DR6, respectively, which may be calculated using the protein-protein interaction prediction server (PPI-Pred) (http://bmbpcu36.leeds.ac.uk/ppi_pred/). From the coordinates of a monomeric protein structure, PPI-Pred typically predicts two or three binding patches or two or three well-defined residue patches that serve as protein-protein interaction sites. In the case of GFD NAPP, PPI-Pred produces two patch predictions (I and II); in the case of the DR6 ectodomain, three predicted interface patches result (I, II and III). The PPI-Pred testing procedure may be validated using the 1sg1 crystal structure.

FIG. 5(b) shows GFD NAPP interface residues derived from the present docking study; FIG. 5(c) shows potential interface residues obtained from the PPI-Pred calculations. Only the calculated PPI-Pred residues that agree with the residues obtained from docking study are shown. These results are summarized in Table 2 below.

TABLE 2
Comparisons between three different methods for predicting
interface residues for GFD NAPP (SEQ ID NO: 1)

ClusPro predicted interface residues for GFD NAPP:
C38, G39, T59, K60, T61, C62, I63, D64, T65, E67, G68, L70,
Q71, Q74, P78, E79, I82, T83, K99, R100, K103,
Q104, E121, F122, V123
PPI-Pred predicted interface residues for GFD NAPP
C38, G39, T61, C62, I63, D64, T65, Q74, P78, E79, I82,
T83, E121, F122, V123
22C11 predicted interface residues for GFD NAPP:
K66, E67, G68, I69, L70, Q71, Y72, C73, Q74, E75, V76,
Y77, P78, E79, L80, Q81
GFD-NAPP amino acid sequence (residues 38-123):
CGRLNMHMNVQNGKWDSDPSGTKTCIDTKEGILQYCQEVY
PELQITNVVEANQPVTIQNWCKRGRKQCKTHPHFVIPYRCLVGEFV

The first row of Table 2 provides the interface residue predictions or contributions of GFD NAPP implied by our DR6-GFD NAPP ClusPro docked model, using a 4.5 Å inter-chain cutoff criterion. The residues provided in the first row provide the basis for comparison with the bottom two rows. Residue agreement with the first row is thus indicated by underlining residues in the bottom two rows. Substantial agreement between the three independently generated data sets verifies our docked model.

The second row of Table 2 provides interface residue predictions for GFD NAPP that are generated using PPI-Pred. Only PPI-Pred residues that agree with the ClusPro residues are shown. For GFD NAPP, PPI-Pred predicted two binding patches (I and II). Patch I has 25 residues; 8 overlap with the ClusPro interface residues; the first 8 residues above correspond to patch I. Patch II has 19 residues; 7 overlap with the ClusPro residues; the last 7 residues are from patch II.

The third row of Table 2 provides interface residue predictions for GFD NAPP that are inferred from the fact that (1) the anti-GFD NAPP antibody 22C11 has a known GFD NAPP binding epitope (displayed) and (2) that 22C11 blocks the interaction between DR6 and GFD NAPP. Thus, we assume or predict that to block the DR6 interaction 22C11 is binding to the very GFD NAPP epitope that, at least in part, mediates binding to DR6. Residues that agree with the ClusPro predictions are underlined. Finally, the fourth row of Table 2 lists the primary amino acid sequence of GFD-NAPP (residues 38-123).

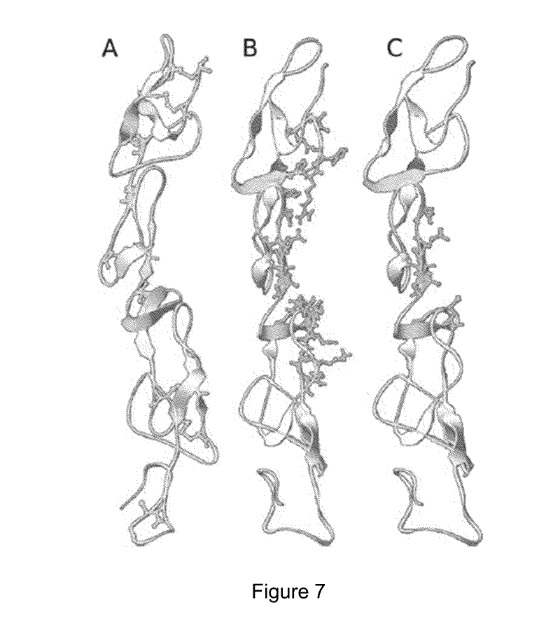

As in the case of GFD NAPP, DR6 interface residues derived from the docking study are compared to potential DR6 interface residues derived from PPI-Pred. Unlike the case with GFD NAPP, an experimentally derived DR6 interface residue set proved to be unavailable. Only the calculated PPI-Pred residues that agree with the residues obtained from docking are shown. The results are summarized in FIG. 7 and in Table 3 below. The evidence indicates that the DR6-GFD NAPP model is of high quality and probable accuracy.

TABLE 3
Comparisons between two different methods for predicting
interface residues for the homology model of the DR6 ectodomain
(SEQ ID NO: 2)

ClusPro predicted interface residues for DR6 ectodomain homology
model: F96, R98, H99, I103, E104, H107, D108, K120, L121, D128,
E130, C131, T132, C139, N141, A142, K158, E162,
T163, E164, D165, R167
PPI-Pred predicted interface residues for DR6 ectodomain homology
model D108, K120, D128, E130, C131, T132, Q139
DR6 amino acid sequence (residues 96-167):
FTRHENGIEKCHDCSQPCPWPMIEKLPCAALTDRECTCPPGMFQS
NATCAPHTVCPVGWGVRKKGTETEDVR

TABLE 4
Physical descriptors and individual binding free energy
contributions for DR6-GFD NAPP interaction

| | Physical | descriptor Free energy contribution |
|---|---|---|
| Physical descriptor (type) | (quantity) | (kcal) |
| −0.79ΔX_{+/-} | −2 | 1.58 |
| 0.075ΔX_{c/s} | −43 | −3.23 |
| −0.65X_{sb} | 4 | −2.6 |
| −0.86X_{hb} | 7 | −6.02 |
| −0.089ΔXₜₒᵣ | −37 | 3.29 |
| −0.00089X_{gap} | 4375 | −3.89 |

Table 4 shows PPI-Pred produced three patch predictions for DR6. Only PPI-Pred predictions that agree with the ClusPro residues are shown. For DR6, PPI-Pred produced three patch predictions (I, II, and III). Patch I is 27 residues, Patch II is 31 residues and patch III is 17 residues. All of the above displayed PPI-Pred residue predictions are derived from Patch II. More details are provided in the text and in Table 1. The good agreement exhibited between the two interface residue data sets verifies the DR6-GFD NAPP docked interaction model. The third row of Table 3 lists the primary amino acid sequence of GFD-NAPP (residues 38-123).

GFD NAPP is 96 residues long (residues 28-123) and consists of 7 beta strands and 1 alpha helix (66-76). The DR6 (TNFR21) ectodomain is 145 residues long (residues 67-211) and is formed by 12 beta strands. It contains 2 N-linked glycosylation sites (ASN82 and ASN 141) and 9 disulfide bonds (residues 67-80, 70-88, 91-106, 109-123, 113-131, 133-144, 150-168, 171-186 and 192-211 of CYS). The disulfide bonds provide structural stability and organize the structure into four cysteine rich domains (CRD) (FIG. 8a). Past work on TNF receptors indicate that the CRDs also mediate protein-protein binding interactions. In the case of DR6, CRD1 includes residues 50-88, CRD2 includes residues 90-131, CRD3 spans residues 133-167, and CRD4 spans residues 170-211. Armed with a structural model of how DR6 interacts with GFD NAPP, we can now rationalize DR6-GFD NAPP binding and consider some implications in terms of these and other structural and energetic categories.

Eq. (1) can be decomposed and each term analyzed to gain insight into the energetic and structural basis of binding (Table 4). The various physical descriptor values provided in Table 4 are well within the ranges established by the physical descriptors derived from known protein-protein interactions. This counts as further evidence that our model is a good one. Overall, the DR6-GFD NAPP interface (FIG. 8a) is characterized by 4 salt bridges (Table 5) and 7 hydrogen bonds (Table 6). In terms of Eq. (1), the hydrogen bonds make a large and stabilizing contribution to binding (≈−6.02 kcal/mol), while the salt bridges make a smaller contribution, especially when the cost of charge group burial is taken into consideration (≈−1.0 kcal/mol). Thus, electrostatic complementarity and hydrogen bonding is seen to play as important a role in DR6-GFD NAPP binding as they do in other TNFR complexes. Thus, the evidence indicates that the gap volume descriptor captures or correlates with stabilizing protein-water-protein interactions across the protein-protein interface. In the case of DR6-GFD NAPP, this favorable contribution to binding is effectively canceled by the unfavorable conformational entropy penalty of binding. Finally, some 43 hydrophobic groups are buried by DR6-GFD NAPP complex formation, contributing ≈−3.2 kcal/mol in binding free energy. This indicates that DR6-GFD NAPP binding is strongly stabilized by the hydrophobic effect. Thus, a decomposition of Eq. (1) indicates that the DR6-GFD NAPP complex is primarily stabilized by hydrogen bonding interactions and the hydrophobic effect.

Eq. (1) can be decomposed and each term analyzed to gain insight into the energetic and structural basis of binding (Table 4). The various physical Equation (1) is used to calculate all predicted binding affinities (kcal/mol). In particular, Eq. (1) is validated by being used to accurately calculate the binding affinity of the p75-NGF interaction and is used to calculate predicted binding affinities for the ten energy optimized ClusPro models. Physical descriptor values and individual binding free energy contributions for the best DR6-GFD NAPP docked model are provided in the table. The predicted binding affinity for the best DR6-GFD NAPP model is calculated to be −11.1 kcal.

The various physical descriptors that contribute to Eq. (1) and their regression-derived weights are listed in column 1. From the top down, the weighted physical descriptors are (1) the change in the number of solvent exposed charged groups, (2) the change in the number of solvent exposed hydrophobic groups (carbon and sulfur atoms), (3) the number of interface salt bridges, (4) the number of interface hydrogen bonds, (5) the change in the number of solvent exposed side chain torsions and (6) the interface gap volume. Not shown is a −0.33 kcal constant contribution.

Column 2 of Table 4 lists the actual descriptor counts and values calculated from the ClusPro generated and energy optimized coordinates of what we identified as the best docked DR6-GFD NAPP model. The units for the gap volume descriptor are Å³.

Column 3 of Table 4 lists the individual free energy contributions implied by the various regression-weighted descriptors. A negative value implies a favorable contribution to binding, while a positive value implies and unfavorable contribution to binding.

TABLE 5
Interface salt bridges for the best predicted DR6-GFD NAPP
docked model

| A | B | Distance (A) |
|---|---|---|
| Glu 104 OE1 | Lys 103 NZ | 3.57 |
| Glu 104 OE2 | Lys 103 NZ | 2.97 |
| Arg 167 NE | Val 123 OXT | 3.73 |
| Arg 167NH1 | Val 123 OXT | 2.91 |

Eq. (1) can be decomposed and each term analyzed to gain insight into the energetic and structural basis of binding (Table 4). The various physical Column A of Table 5 refers to the DR6 homology model (receptor) and column B refers to the GFD-NAPP crystal structure (ligand). A salt bridge is defined as an interaction between two charged atoms that are separated by 4.0 Å or less.

TABLE 6
Interface hydrogen bonds for the best predicted DR6-GFD NAPP
docked model

| A | B |
|---|---|
| Cys 131 O | Gln 71 OE1 |
| Thr 132 OG1 | Gln 71 NE2 |
| Thr 132 OG1 | Gln 71 OE1 |
| Gln 139 OE1 | Gln 74 OE1 |
| Arg 98 NH1 | Cys 62 O |
| Thr 163 OG1 | Ile 82 O |
| Arg 167 NH1 | Val 123 OXT |

Column A of Table 6 refers to the DR6 homology model (receptor) and column B refers to the GFD-NAPP crystal structure (ligand). Hydrogen bonds are identified according to the “Levitt” criteria, as implemented in a hydrogen bond detection program. An exemplary embodiment of such a program is available as part of the “Libproteingeometry” distribution and is available at the Gerstein lab page (http://geometry.molmovdb.org/files/libproteingeometry/src-prog2/README.htm). The same program is used to calculate the hydrogen bond descriptor of Eq. (1). Due to the difficulty of distinguishing nitrogen from oxygen atoms, Gln and Asn epsilon (OE1) and delta (OD1) oxygen atoms are counted as hydrogen bond donors and acceptors, respectively.

The GFD NAPP alpha helix plays a key energetic role in DR6 recognition. Based on the model for DR6-GFD NAPP, CRD2 and CRD3 do indeed mediate DR6 binding to GFD NAPP. In particular, the CRD2-CRD3 junction forms a shallow depression or groove that perfectly accommodates the lone GFD NAPP alpha helix (66-76) as it is shown in FIG. 8a. In terms of atomic interactions, the GFD helix packs against the disulfide-bridge stabilized DR6 beta-strand 130-132. Gln 71 of the GFD NAPP helix forms a total of three stabilizing hydrogen bonding interactions with Cys 131 and Thr 132 of the DR6 beta strand, see FIG. 8b and FIG. 8c box 6. Gln 74 of the GFD NAPP alpha helix also forms an apparent hydrogen bond with Gln 139 of DR6, see FIG. 8b and FIG. 8c box 2. Indeed, calculations made using Eq. (1) indicate that the GFD NAPP helix plays an important recognition role, supplying ≈−5.0 kcal/mol in binding free energy or roughly 45% of the total binding affinity. Thus, the GFD alpha helix plays a key role in DR6 binding, in particular, as a recognition motif for DR6 strand 130-132. In all, the data indicates that CRD2 and CRD3 disulfide bridges 113-131 and 133-144 orient and stabilize DR6 beta strand 130-132 for thermodynamically favorable binding to GFD NAPP helix 66-76.

A network of two salt bridges and a single hydrogen bond is formed between the GFD NAPP C-terminal carboxyl group of Val 123 and the side chain of Arg 167 of DR6 as shown in FIG. 8c box 5. This interaction is of special interest given that Val 123 occupies different positions in the unbound but high resolution 1mwp GFD NAPP structure and the dimeric but lower resolution 3ktm NAPP structure. Thus, the flexibility of the GFD NAPP C-terminus is seen to be important in DR6 recognition.

Another interesting set of atomic interactions include hydrogen bonding interactions between the DR6 side chain of Thr 163 and the GFD NAPP main chain of Ile 82, on the one hand, and the DR6 side chain of Arg 98 and the GFD NAPP main chain of Cys 62, on the other hand, see FIG. 8b and FIG. 8c box 4 and box 3 respectively. The interactions are interesting because they occur on opposite sides of the GFD NAPP helix, with the former interactions positioned at the ‘front’ of the helix and the latter positioned toward the ‘rear’ of the helix as if they exist to correctly position and orient the GFD NAPP helix to interact with the DR6 130-132 beta strand. Indeed, the two salt bridges formed between the side chains of GFD NAPP Lys 103 and DR6 Glu 104 (see FIG. 8c box 1), on one hand, and the previously described network of interactions between Val 123 and Arg 167, on the other, also flank either side of the GFD NAPP alpha helix and exist in a ‘front-to-back’ orientation, thus, reinforcing the indication that interactions outside of the GFD NAPP alpha helix helps position it into close and stabilizing contact with the disulfide bridge stabilized DR6 beta strand 130-132.

Compared to other TNFR crystallographic binding configurations, such as p75-NGF (TNFR16-NGF) and DR5-TRAIL (TNFR10-TRAIL), the rigid-body binding orientation of the DR6-GFD NAPP interaction is unique (FIG. 8d). The GFD NAPP ligand is also unique among TNFR ligands in that it is monomeric and includes alpha helical structure. In addition, the GFD NAPP alpha helix plays an important role in providing the binding energy that drives DR6 recognition. Given that NAPP fails to bind other TNFR receptors with high affinity and given the uniqueness of its alpha helical motif, the GFD NAPP alpha helix confers specificity to DR6 binding.

Recent work shows that the extracellular part of the DR6 molecule is highly N- and O-glycosylated. Such post-translational modifications can influence folding, transport and function of the receptor. Glycosylation (in particular, N-linked glycosylation) could also directly regulate the affinity of the DR6 receptor for NAPP by modulating DR6-GFD NAPP interface contacts. An analysis of the available solvent-exposed ectodomain DR6ASN residues (which is cross-checked at ExPASy [http://expasy.org] or another structural bioinformatics program or website), revealing that the solvent exposed ASN residues (ASN82 and ASN141 of the DR6, see FIG. 8a) are probably too far away from and oriented away from the key DR6-GFD NAPP interface residues. Hence, our model indicates that glycosylation does not directly modulate DR6-NAPP binding. This, however, does not mean that glycosylation does not affect binding; it affects binding through some other mechanism.

The DR6-GFD NAPP binding reaction approximates rigid-body association and the unbound DR6 conformation is only weakly stabilized with respect to the bound conformation. The neurotrophin p75 receptor binds NGF with a free energy of binding (ΔG_{bind,exp}) of −12.4 kcal/mol. Applying Eq. (1) to the 1sg1 p75-NGF coordinates yielded an estimated ΔG_{bind,empirical}≈−11.8 kcal/mol. This helps validate the use of Eq. (1) on DR6. Perhaps more importantly, given that Eq. (1) is only accurate to within ≈1.0-1.5 kcal/mol for binding reactions that approximate rigid-body association, this strongly indicates that the p75-NGF binding reaction approximates rigid-body association. This inference is further strengthened by the fact that the structure of unbound murine NGF can be superimposed to within ≈1.0 Å Ca root-mean-square-deviations (rmsd) of the common amino acids of the 1sg1 structure of bound human NGF. Eq. (1) is also used to accurately estimate the binding affinity of the DR6-GFD NAPP binding reaction. Moreover, as described above the bound and unbound conformations of GFD NAPP are nearly identical. Thus, the DR6-GFD NAPP reaction accurately approximates rigid-body association. Therefore, the unbound or native state conformation of DR6 (and p75) is stable with respect to the bound conformation by ≈1.0-1.5 kcal/mol.

A structurally modest binding associated acquisition of beta structure in DR6 residues 130-132 is seen to provide a conformational switch for cellular apoptosis. p75 and DR6 binding probably approximate rigid-body association and their unbound conformations are 1.0-1.5 kcal/mol stable with respect to their bound conformations. For rigid-body association, our published and unpublished work indicates that binding associated conformational changes with respect to unbound conformations are typically <1.5 Å Ca rmsd. Thus, the binding associated p75 and DR6 structural transitions likely involves modest conformational rearrangements on the order of 1.5 Ca Å rmsd. This inference is consistent with the modest p75-NGF binding associated gain in secondary structure indicated by a spectroscopic analysis of p75-NGF binding.

Our experiments indicate that DR6 residues 130-132 are the primary residues that undergo a modest binding associated reorganization from a relatively unstructured state to a beta-structured bound state. All of this indicates that NAPP binding to DR6 mediates cellular apoptosis through a structurally subtle acquisition of beta structure in DR6 residues 130-132.

The unbound (pro-life) DR6 conformation is only weakly stabilized with respect to the DR6 bound (pro-death) conformation, which indicates spontaneous apoptosis for a non-trivial number of DR6-sensitive cells. Because ligand binding to p75 and DR6 leads to apoptosis through caspase mediation, we can refer to the unbound conformations as “pro-life” conformations and the bound conformations as “pro-death” conformations. As discussed previously, it can be argued that rigid-body association for p75 and DR6 indicates that their unbound conformations (pro-life conformations) are only weakly stabilized with respect to their bound (pro-death conformations) conformations 1.0-1.5 kcal/mol) and are structurally similar, at least in terms of Ca rmsd calculations. Thus, in the absence of pro-death signaling (no GFD NAPP) and for purely ‘intrinsic” biophysical reasons, we expect roughly 8.0%-16.0% of DR6 (and p75) receptors to spontaneously adopt subtly different pro-death conformations and, in the absence of some other mechanism, to spontaneously engage the cell's apoptotic machinery, culminating in cell death. Thus, in the absence of some other mechanism, a non-trivial fraction of DR6 (and p75) sensitive cells are expected to fall victim to spontaneous apoptosis through a subtle change in ectodomain conformation and in the absence of ligand binding. It is worth noting that this is consistent with what is known about the roughly 15% of neutrophil cells that undergo spontaneous apoptosis through caspase mediation in the absence of ligand-death receptor binding.

Dialysis and gel experiments indicate that the addition of a small concentration of Cu2+ ions to the buffer solution (about 40 micromolar) is enough to increase N-APP affinity to DR6. A portion of the copper binding domain (CuBD) located in the E1 domain of N-APP undergoes a conformational change upon Cu2+ binding. Wang Q, Werstiuk N H, Kramer J R, Bell R A. Effects of Cu ions and explicit water molecules on the copper binding domain of amyloid precursor protein APP(131-189): a molecular dynamics study. J Phys Chem B. 2011 Jul. 28; 115(29):9224-35. Epub 2011 Jun. 29. Experimental study indicates that the E2 domain of the N-APP undergoes large conformational changes upon binding of Cu2+ and Zn2+. Dahms S O, Könnig I, Roeser D, Gührs K H, Mayer M C, Kaden D, Multhaup G, Than M E. X-ray structure of the E2 domain of the human amyloid precursor protein (APP) in complex with zinc. J Mol Biol. 2012 Feb. 24; 416(3):438-52. Epub 2012 Jan. 4. Such conformational transitions may be responsible for the increased affinity of N-APP to DR6 and may be utilized accordingly. Considering all of the above and the newly available crystal structures of DR6, E1 and E2 domains of N-APP, a detailed computational investigation of the N-APP-DR6 interaction is warranted. Dahms S O, Hoefgen S, Roeser D, Schlott B, Gührs K H, Than M E. Structure and biochemical analysis of the heparin-induced E1 dimer of the amyloid precursor protein. Proc Natl Acad Sci USA. 2010 Mar. 23; 107(12):5381-6. Epub 2010 Mar. 8. Kuester M, Kemmerzehl S, Dahms S O, Roeser D, Than M E. The crystal structure of death receptor 6 (DR6): a potential receptor of the amyloid precursor protein (APP). J Mol Biol. 2011 Jun. 3; 409(2):189-201. Epub 2011 Apr. 2.

Homology modeling is used to predict the 3D-structure of a unknown protein based on the known structure of a similar protein. During evolution, sequence changes much faster than structure. It is therefore possible to identify the 3D-structure by looking at a molecule with some sequence homology or identity. It has been described how much sequence homology and identity is needed with a certain number of aligned residues to reach the safe homology modeling zone. (See, for example, Marketa Zvelebil & Jeremy Baum, Understanding Bioinformatics (Garland Science 2007)). For example, for a sequence of approximately 100 residues, a sequence homology of 30% is a conservative number for structure prediction. Of course, each model varies and in many examples significantly less homology is needed to accurately predict structures in this amino acid residue length. When the sequence identity and/or homology falls in the safe homology modeling zone, we can assume that the 3D-structure of both sequences is roughly the same.

Our model provides a structural basis for future experimental testing and possible refinement. For example, interface mutagenesis experiments could be run to test and possibly refine the model. The model also provides a basis for designing experiments to test other DR6 and GFD NAPP related hypotheses and to possibly rationalize data that we have failed to consider. Perhaps most importantly, the model can be used in structure-based design studies aimed at identifying drug-like compounds to modulate DR6-GFD NAPP binding and treat AD.

The present invention provides compositions and methods for discovering molecules that have the potential to interfere with the DR6-GFD NAPP interaction, thus treating or ameliorating AD.

In one aspect, the present invention is directed towards a polypeptide whose amino acid residues have about 30% homology to residues 38-123 of the growth factor-like domain (GFD) of the N-terminal APP fragment (NAPP). The polypeptide adopts a specific conformation in vivo characterized by having seven beta strands. In addition, the residues 66-81 of the polypeptide adopt a lone alpha-helix motif. Finally, residue 62 of the polypeptide is Cysteine, residue 71 is Glutamine, residue 74 is Glutamine, residue 82 is Isoleucine, residue 103 is Lysine, and residue 123 is Valine. In another aspect, the present invention is directed towards a polypeptide whose amino acid residues have about 40% homology to residues 38-123 of the growth factor-like domain (GFD) of the N-terminal APP fragment (NAPP). In another aspect, the present invention is directed towards a polypeptide whose amino acid residues have about 50% homology to residues 38-123 of the growth factor-like domain (GFD) of the N-terminal APP fragment (NAPP). In another aspect, the present invention is directed towards a polypeptide whose amino acid residues have about 75% homology to residues 38-123 of the growth factor-like domain (GFD) of the N-terminal APP fragment (NAPP). In another aspect, the present invention is directed towards a polypeptide whose amino acid residues have about 90% homology to residues 38-123 of the growth factor-like domain (GFD) of the N-terminal APP fragment (NAPP). In another aspect, the present invention is directed towards a polypeptide whose amino acid residues have about 100% homology to residues 38-123 of the growth factor-like domain (GFD) of the N-terminal APP fragment (NAPP).

In another aspect, the present invention is directed towards a polypeptide whose amino acid residues have about 30% homology to residues 96-167 of Death Cell Receptor 6 (DR6), including a first Cysteine Rich Domain (CRD) with at least 30% homology to amino acid residues 96 to 131 of DR6 and a second Cysteine Rich Domain (CRD) with at least 30% homology to amino acid residues 133 to 167 of DR6. The polypeptide adopts a specific conformation in vivo characterized by having twelve beta strands. In addition, residue 98 of the polypeptide is Arginine, residue 104 is Glutamic Acid, residue 131 is Cysteine, residue 132 is Threonine, residue 139 is Glutamine, residue 163 is Threonine, and residue 167 is Arginine. In other aspects, the invention is directed to such a polypeptide whose amino acid residues have about 40%, homology to residues 96-167 of the DR6. In other aspects, the invention is directed to such a polypeptide whose amino acid residues have about 40% homology to residues 96-167 of the DR6. In other aspects, the invention is directed to such a polypeptide whose amino acid residues have about 50% homology to residues 96-167 of the DR6. In other aspects, the invention is directed to such a polypeptide whose amino acid residues have about 75% homology to residues 96-167 of the DR6. In other aspects, the invention is directed to such a polypeptide whose amino acid residues have about 90% homology to residues 96-167 of the DR6. In other aspects, the invention is directed to such a polypeptide whose amino acid residues have about 100% homology to residues 96-167 of the DR6.

The calculated free energy of binding between the predicted interface residues of our DR6-GFD NAPP model of approximately −11.1 kcal/mol, this affinity corresponds to a binding affinity in the nanomolar range. This supports the proposition of a polypeptide, peptidomimetic, or small molecule with a binding affinity of about 500 nM or less for use in contacting the surface of DR6 and/or NAPP.

In another aspect, the present invention is directed toward methods for screening chemical compounds to determine their potential to modulate or bind to DR6 to prevent or inhibit its binding to GFD NAPP or to bind to GFD NAPP to prevent or inhibit its binding to DR6. In still another aspect, the present invention is directed toward methods for screening chemical compounds to determine their potential to treat, ameliorate or retard the onset of AD These methods utilize the polypeptides described above.

Assays for Evaluating Compounds Designed to Modulate the Interaction of DR6 and GFD NAPP.

A variety of methods for modulating the interaction of DR6-GFD NAPP using modulator compounds are contemplated by the present invention. As used herein, the tem “modulator” or “modulator compound” is intended to mean a peptide, polypeptide, small molecule or other chemical compound that interferes with or prevents the binding of DR6 and GFD NAPP to each other.

In one aspect, the present invention provides methods of screening the subject druggable regions of DR6 and/or GFD NAPP to discover potential modulator compounds, as well as methods of designing such modulators. Modulators to the polypeptides of the invention and other structurally related molecules, and complexes containing the same, is identified and developed as set forth below and otherwise using techniques and methods known to those of skill in the art. The modulators of the invention may be employed, for instance, to treat, ameliorate, or retard the progression of AD or other neurological conditions.

In one aspect, the present invention is directed towards a modulator compound that interacts with the subject druggable regions so as to reduce or prevent the binding of GFD NAPP and DR6 to each other. Such modulators may in certain embodiments interact with a druggable region of the invention. In still another aspect, the present invention is directed toward a modulator that is a fragment (or homolog of such fragment or mimetic of such fragment) of the druggable region of GFD-NAPP and/or DR6 and competes with that druggable region for binding with DR6 or GFD NAPP, as applicable. Modulators of any of the above-described druggable regions may be used alone or in complementary approaches to treat, ameliorate, or retard the progression of AD or other neurological conditions.

For example, in one aspect, the present invention contemplates a method for treating a patient suffering from AD or other neurological condition comprising administering to the patient an amount of a compound identified by a method of the present invention that is effective to reduce or prevent the binding of DR6 to GFD NAPP. The present invention further contemplates a method for treating a subject suffering from AD or other neurological condition, comprising administering to a patient having the condition a therapeutically effective amount of a molecule identified using one of the methods of the present invention.

In another embodiment, the compounds discussed above may be used in the manufacture of a medicament for any number of uses, including, for example, treating any disease or other treatable condition of a patient mediated by DR6 binding to GFD NAPP.

A number of techniques can be used to screen, identify, select and design chemical entities capable of interfering with the binding of DR6 to GFD NAPP. Knowledge of the structure of DR6 and/or GFD NAPP, determined in accordance with the methods described herein, permits the design and/or identification of molecules and/or other modulators which have a shape complementary to the conformation of DR6 and/or GFD NAPP, or more particularly, a druggable region thereof. It is understood that such techniques and methods may use, in addition to the exact structural coordinates and other information for DR6 and GFD NAPP, structural equivalents thereof described above (including, for example, those structural coordinates that are derived from the structural coordinates of amino acids contained in a druggable region as described above).

The term “chemical entity,” as used herein, refers to chemical compounds, complexes of two or more chemical compounds, and fragments of such compounds or complexes. In certain instances, it is desirable to use chemical entities exhibiting a wide range of structural and functional diversity, such as compounds exhibiting different shapes (e.g., flat aromatic rings(s), puckered aliphatic rings(s), straight and branched chain aliphatics with single, double, or triple bonds) and diverse functional groups (e.g., carboxylic acids, esters, ethers, amines, aldehydes, ketones, and various heterocyclic rings). Such chemical entities may be peptides, polypeptides, or non-peptide chemical compounds.

In one aspect, the method of drug design generally includes computationally evaluating the potential of a selected chemical entity to associate with any of the molecules or complexes of the present invention (or portions thereof). For example, this method may include the steps of (a) employing computational means to perform a fitting operation between the selected chemical entity and a druggable region of the molecule or complex; and (b) analyzing the results of said fitting operation to quantify the association between the chemical entity and the druggable region.

A chemical entity may be examined either through visual inspection or through the use of computer modeling using a docking program, representative embodiments which include GRAM, DOCK, or AUTODOCK. This procedure can include computer fitting of chemical entities to a target to ascertain how well the shape and the chemical structure of each chemical entity will complement or interfere with the structure of DR6 and/or GFD NAPP so as to be likely to prevent or inhibit DR6 binding to GFD NAPP. Computer programs may also be employed to estimate the attraction, repulsion, and steric hindrance of the chemical entity to a druggable region, for example. Generally, the tighter the fit (e.g., the lower the steric hindrance, and/or the greater the attractive force), the more potent the chemical entity will be to prevent or inhibit the binding of DR6 to GFD NAPP because these properties are consistent with a tighter binding constant. Furthermore, the greater the specificity in the design of a chemical entity, the more likely it becomes that the chemical entity will not interfere with related proteins, which may minimize potential side-effects due to unwanted interactions.

A variety of computational methods for molecular design, in which the steric and electronic properties of druggable regions are used to guide the design of chemical entities, are known. Representative methods are described in: Cohen et al. (1990) J. Med. Cam. 33: 883-894; Kuntz et al. (1982) J. Mol. Biol. 161: 269-288; DesJarlais (1988) J. Med. Cam. 31: 722-729; Bartlett et al. (1989) Spec. Publ., Roy. Soc. Chem. 78: 182-196; Goodford et al. (1985) J. Med. Cam. 28: 849-857; and Desjarlais et al. J. Med. Cam. 29: 2149-2153. There are many known directed methods known in the art. One example includes design by analogy, in which 3-D structures of known chemical entities (such as from a crystallographic database) are docked to the druggable region and scored for goodness-of-fit. Another example includes de novo design, in which the chemical entity is constructed piece-wise in the druggable region. The chemical entity may be screened as part of a library or a database of molecules. Databases which may be used include those of ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions.

Chemical entities may be tested for their capacity to fit spatially with a druggable region or other portion of DR6 and/or GFP NAPP. As used herein, the term “fit(s) spatially” means that the three-dimensional structure of the chemical entity is accommodated geometrically by a druggable region. A favorable geometric fit occurs when the surface area of the chemical entity is in close proximity with the surface area of the druggable region without forming unfavorable interactions. A favorable complementary interaction occurs when the chemical entity interacts by hydrophobic, aromatic, ionic, dipolar, or hydrogen donating and accepting forces. Unfavorable interactions may, for example, be steric hindrance between atoms in the chemical entity and atoms in the druggable region.

For the electronic embodiment of the present invention, the chemical entities may be positioned in a druggable region through computational docking. For the structural embodiment of the present invention, the chemical entities may be positioned in the druggable region by, for example, manual docking. As used herein the term “docking” refers to a process of placing a chemical entity in close proximity with a druggable region, or a process of finding low energy conformations of a chemical entity/druggable region complex.

In an illustrative embodiment, the design of a potential modulator begins from the general perspective of shape complimentary for the druggable region of DR6 and/or GFD NAPP, and a search algorithm is employed which is capable of scanning a database of small molecules of known three-dimensional structure for chemical entities which fit geometrically with the target(s) druggable region(s). Most algorithms of this type provide a method for finding a wide assortment of chemical entities that are complementary to the shape of a druggable region of DR6 and/or GFD NAPP. Each of a set of chemical entities from a particular data-base, such as the Cambridge Crystallographic Data Centre (CCDC) (http://www.ccdc.cam.ac.uk/), is individually docked to the druggable region of DR6 and/or GFD NAPP in a number of geometrically permissible orientations with use of a docking algorithm. In certain embodiments, a set of computer algorithms called DOCK, can be used to characterize the shape of invaginations and grooves that form the active sites and recognition surfaces of the druggable region. The program can also search a database of small molecules for templates whose shapes are complementary to particular binding sites of DR6 and/or GFD NAPP.

The orientations are evaluated for goodness-of-fit and the best are kept for further examination using molecular mechanics programs, such as AMBER or CHARMM. Such algorithms have previously proven successful in finding a variety of chemical entities that are complementary in shape to a druggable region.

Goodford (1985, J Med Chem 28:849-857) and Boobbyer et al. (1989, J Med Chem 32:1083-1094) have produced a computer program (GRID) which seeks to determine regions of high affinity for different chemical groups (termed probes) of the druggable region. GRID provides a tool for indicating modifications to known chemical entities that enhance binding. It may be anticipated that some of the sites discerned by GRID as regions of high affinity correspond to “pharmacophoric patterns” determined inferentially from a series of known ligands. As used herein, a “pharmacophoric pattern” is a geometric arrangement of features of chemical entities that is believed to be important for binding. Attempts have been made to use pharmacophoric patterns as a search screen for novel ligands (Jakes et al. (1987) J Mol Graph 5:41-48; Brint et al. (1987) J Graph 5:49-56; Jakes et al. (1986) J Mol Graph 4:12-20).

Yet a further embodiment of the present invention utilizes a computer algorithm such as CLIX which searches such databases as CCDC for chemical entities which can be oriented with the druggable region of DR6 and/or GFD NAPP in a way that is both sterically acceptable and has a high likelihood of achieving favorable chemical interactions between the chemical entity and the surrounding amino acid residues. The method is based on characterizing the region in terms of an ensemble of favorable binding positions for different chemical groups and then searching for orientations of the chemical entities that cause maximum spatial coincidence of individual candidate chemical groups with members of the ensemble. The algorithmic details of CLIX is described in Lawrence et al. (1992) Proteins 12:3141.

In this way, the efficiency with which a chemical entity may bind to or interfere with a druggable region may be tested and optimized by computational evaluation. For example, for a favorable association with a druggable region, a chemical entity should preferably demonstrate a relatively small difference in energy between its bound and free states (i.e., a small deformation energy of binding). Thus, certain, more desirable chemical entities will be designed with a deformation energy of binding of not greater than about 7 kcal/mole, and more preferably, not greater than 5 kcal/mole. Chemical entities may interact with a druggable region in more than one conformation that is similar in overall binding energy. In those cases, the deformation energy of binding is taken to be the difference between the energy of the free entity and the average energy of the conformations observed when the chemical entity binds to the target.

In this way, the present invention provides computer-assisted methods for identifying or designing a potential modulator of the binding of DR6 to GFD NAPP including: supplying a computer modeling application with a set of structure coordinates of a molecule or complex, the molecule or complex including at least a portion of a druggable region from DR6 and/or GFD NAPP; supplying the computer modeling application with a set of structure coordinates of a chemical entity; and determining whether the chemical entity is expected to bind to the molecule or complex, wherein binding to the molecule or complex is indicative of potential modulation of the binding of DR6 and GFD NAPP to each other.

In another aspect, the present invention provides a computer-assisted method for identifying or designing a potential modulator to DR6 and/or GFD NAPP, supplying a computer modeling application with a set of structure coordinates of a molecule or complex, the molecule or complex including at least a portion of a druggable region of DR6 and/or GFD NAPP; supplying the computer modeling application with a set of structure coordinates for a chemical entity; evaluating the potential binding interactions between the chemical entity and active site of the molecule or molecular complex; structurally modifying the chemical entity to yield a set of structure coordinates for a modified chemical entity, and determining whether the modified chemical entity is expected to bind better or less well to the molecule or complex, wherein binding to the molecule or complex is indicative of potential prevention or interference of the binding of DR6 and GFD NAPP.

In one embodiment, a potential modulator can be obtained by screening a peptide or other compound or chemical library (Scott and Smith, Science, 249:386-390 (1990); Cwirla et al., Proc. Natl. Acad. Sci., 87:6378-6382 (1990); Devlin et al., Science, 249:404-406 (1990)). A potential modulator selected in this manner could then be systematically modified by computer modeling programs until one or more promising potential drugs are identified. Such analysis has been shown to be effective in the development of HIV protease modulators (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)). Alternatively a potential modulator may be selected from a library of chemicals such as those that can be licensed from third parties, such as chemical and pharmaceutical companies. A third alternative is to synthesize the potential modulator de novo.

For example, in certain embodiments, the present invention provides a method for making a potential modulator of DR6 and/or GFD NAPP, the method including synthesizing a chemical entity or a molecule that prevents or inhibits the binding of DR6 and GFD NAPP, the chemical entity having been identified during a computer-assisted process comprising: 1) supplying a computer modeling application with a set of structure coordinates of a molecule or complex, the molecule or complex including at least one druggable region from DR6 and/or GFD NAPP; 2) supplying the computer modeling application with a set of structure coordinates of a chemical entity; and 3) determining whether the chemical entity is expected to bind to the molecule or complex at the active site, wherein binding to the molecule or complex is indicative of potential activity to inhibit or prevent the binding of DR6 and GFD NAPP. This method may further include the steps of evaluating the potential binding interactions between the chemical entity and the active site of the molecule or molecular complex and structurally modifying the chemical entity to yield a set of structure coordinates for a modified chemical entity, which steps may be repeated one or more times.

Once a potential modulator is identified, it can then be physically tested in any standard assay, as is well-understood in the art. Further refinements to the structure of the modulator will generally be necessary and can be made by the successive iterations of any and/or all of the steps provided by the particular screening assay, in particular further structural analysis by e.g., ¹⁵N NMR relaxation rate determinations or x-ray crystallography with the modulator bound to DR6 and/or GFD NAPP. These studies may be performed in conjunction with biochemical assays.

Once identified, a potential modulator compound may be used as a model structure, and analogs to the compound can be obtained. The analogs may then be screened for their ability to bind to DR6 and/or GFD NAPP. An analog of the potential modulator is chosen as a modulator when it binds to DR6 and/or GFD NAPP with a higher binding affinity than the predecessor modulator. As further described below, this process may be performed iteratively.

In a related approach, iterative drug design may be used to identify modulators of DR6 and/or GFD NAPP. Iterative drug design is a method for optimizing associations between a protein and a modulator by determining and evaluating the three dimensional structures of successive sets of protein/modulator complexes. In iterative drug design, crystals of a series of protein/modulator complexes are obtained and the three-dimensional structures of each complex is solved. Such an approach provides insight into the association between the proteins and modulators of each complex. For example, this approach may be accomplished by selecting modulators with modulatory activity, obtaining crystals of this new protein/modulator complex, solving the three dimensional structure of the complex, and comparing the associations between the new protein/modulator complex and previously solved protein/modulator complexes. By observing how changes in the modulator affected the protein/modulator associations, these associations may be optimized.

In addition to designing and/or identifying a chemical entity to associate with a druggable region, as described above, the same techniques and methods may be used to design and/or identify chemical entities that either associate, or do not associate, with affinity regions, selectivity regions or undesired regions of protein targets. By such methods, selectivity for one or a few targets, or alternatively for multiple targets, from the same species or from multiple species, can be achieved.

For example, a chemical entity may be designed and/or identified for which the binding energy for one druggable region, e.g., an affinity region or selectivity region, is more favorable than that for another region, e.g., an undesired region, by about 20%, 30%, 50% to about 60% or more. It may be the case that the difference is observed between (a) more than two regions, (b) between different regions (selectivity, affinity or undesirable) from the same target, (c) between regions of different targets, (d) between regions of homologs from different species, or (e) between other combinations. Alternatively, the comparison may be made by reference to the Kd, usually the apparent Kd, of said chemical entity with the two or more regions in question.

In another aspect, prospective modulators are screened for binding to two nearby druggable regions on a target protein. For example, a modulator that binds a first region of a target polypeptide does not bind a second nearby region. Binding to the second region can be determined by monitoring changes in a different set of amide chemical shifts in either the original screen or a second screen conducted in the presence of a modulator (or potential modulator) for the first region. From an analysis of the chemical shift changes, the approximate location of a potential modulator for the second region is identified. Optimization of the second modulator for binding to the region is then carried out by screening structurally related compounds (e.g., analogs as described above). When modulators for the first region and the second region are identified, their location and orientation in the ternary complex can be determined experimentally. On the basis of this structural information, a linked compound, e.g., a consolidated modulator, is synthesized in which the modulator for the first region and the modulator for the second region are linked. In certain embodiments, the two modulators are covalently linked to form a consolidated modulator. This consolidated modulator may be tested to determine if it has a higher binding affinity for the target than either of the two individual modulators. A consolidated modulator is selected as a modulator when it has a higher binding affinity for the target than either of the two modulators. Larger consolidated modulators can be constructed in an analogous manner, e.g., linking three modulators which bind to three nearby regions on the target to form a multilinked consolidated modulator that has an even higher affinity for the target than the linked modulator. In this example, it is assumed that is desirable to have the modulator bind to all the druggable regions. However, it may be the case that binding to certain of the druggable regions is not desirable, so that the same techniques may be used to identify modulators and consolidated modulators that show increased specificity based on binding to at least one but not all druggable regions of a target.

The present invention provides a number of methods that use drug design as described above. For example, in one aspect, the present invention contemplates a method for designing a candidate compound for screening for modulators that would inhibit or prevent binding of DR6 to GFD NAPP, the method comprising: (a) determining the three dimensional structure of a crystallized DR6 and/or GFD NAPP protein or a fragment thereof; and (b) designing a candidate modulator based on the three dimensional structure of the crystallized polypeptide or fragment.

In another aspect, the present invention contemplates a method for identifying a potential modulator of DR6 and/or GFD NAPP, the method comprising: (a) providing the three-dimensional coordinates of DR6 and/or GFD NAPP or a fragment thereof; (b) identifying a druggable region of the polypeptide or fragment; and (c) selecting from a database at least one compound that comprises three dimensional coordinates which indicate that the compound may bind the druggable region.

In another aspect, the present invention contemplates a method for identifying a potential modulator of a molecule comprising a druggable region having homology to the alpha helix of residues 66-81 of GFD NAPP, the method comprising: (a) using the atomic coordinates of said amino acid residues, +/−a root mean square deviation from the backbone atoms of the amino acids of not more than 1.5 Angstrom, to generate a three-dimensional structure of the druggable region; (b) employing the three dimensional structure to design or select the potential modulator; (c) synthesizing the modulator; and (d) contacting the modulator with the molecule to determine the ability of the modulator to interact with the molecule.

In another aspect, the present invention contemplates an apparatus for determining whether a compound is a potential modulator of DR6 and/or GFD NAPP, the apparatus comprising: (a) a memory that comprises: (i) the three dimensional coordinates and identities of the atoms of DR6 and/or GFD NAPP or a fragment thereof that form a druggable site; and (ii) executable instructions; and (b) a processor that is capable of executing instructions to: (i) receive three-dimensional structural information for a candidate compound; (ii) determine if the three-dimensional structure of the candidate compound is complementary to the structure of the druggable site; and (iii) output the results of the determination.

The synthesis and screening of combinatorial libraries is a well-known strategy for the identification of organic molecules having potential to bind to a biological target of interest. According to the present invention, the synthesis of libraries containing molecules that bind, interact with, or modulate the activity/function of DR6 and/or GFD NAPP may be performed using established combinatorial methods for solution phase, solid phase, or a combination of solution phase and solid phase synthesis techniques. The synthesis of combinatorial libraries is well known in the art and has been reviewed (see, e.g., “Combinatorial Chemistry”, Chemical and Engineering News, Feb. 24, 1997, p. 43; Thompson et al., Chem. Rev. (1996) 96:555). Many libraries are commercially available. One of ordinary skill in the art will realize that the choice of method for any particular embodiment will depend upon the specific number of molecules to be synthesized, the specific reaction chemistry, and the availability of specific instrumentation, such as robotic instrumentation for the preparation and analysis of the inventive libraries. In certain embodiments, the reactions to be performed to generate the libraries are selected for their ability to proceed in high yield, and in a stereoselective and regioselective fashion, if applicable.

In one aspect of the present invention, the inventive libraries are generated using a solution phase technique. Traditional advantages of solution phase techniques for the synthesis of combinatorial libraries include the availability of a much wider range of reactions, and the relative ease with which products may be characterized, and ready identification of library members, as discussed below. For example, in certain embodiments, for the generation of a solution phase combinatorial library, a parallel synthesis technique is utilized, in which all of the products are assembled separately in their own reaction vessels. In a particular parallel synthesis procedure, a microlitre plate containing n rows and m columns of tiny wells which are capable of holding a few milliliters of the solvent in which the reaction will occur, is utilized. It is possible to then use n variants of reactant A, such as a ligand, and m variants of reactant B, such as a second ligand, to obtain n.times.m variants, in n.times.m wells. One of ordinary skill in the art will realize that this particular procedure is most useful when smaller libraries are desired, and the specific wells may provide a ready means to identify the library members in a particular well.

In other embodiments of the present invention, a solid phase synthesis technique is utilized. Solid phase techniques allow reactions to be driven to completion because excess reagents may be utilized and the unreacted reagent washed away. Solid phase synthesis also allows the use a technique called “split and pool”, in addition to the parallel synthesis technique, developed by Furka. See, e.g., Furka et al., Abstr. 14th Int. Congr. Biochem., (Prague, Czechoslovakia) (1988) 5:47; Furka et al., Int. J. Pept. Protein Res. (1991) 37:487; Sebestyen et al., Bioorg. Med. Chem. Lett. (1993) 3:413. In this technique, a mixture of related molecules may be made in the same reaction vessel, thus substantially reducing the number of containers required for the synthesis of very large libraries, such as those containing as many as or more than one million library members. As an example, the solid support with the starting material attached may be divided into n vessels, where n represents the number species of reagent A to be reacted with the such starting material. After reaction, the contents from n vessels are combined and then split into m vessels, where m represents the number of species of reagent B to be reacted with the now modified starting materials. This procedure is repeated until the desired number of reagents is reacted with the starting materials to yield the inventive library.

The use of solid phase techniques in the present invention may also include the use of a specific encoding technique. Specific encoding techniques have been reviewed by Czarnik in Current Opinion in Chemical Biology (1997) 1:60. One of ordinary skill in the art will also realize that if smaller solid phase libraries are generated in specific reaction wells, such as 96 well plates, or on plastic pins, the reaction history of these library members may also be identified by their spatial coordinates in the particular plate, and thus are spatially encoded. In other embodiments, an encoding technique involves the use of a particular “identifying agent” attached to the solid support, which enables the determination of the structure of a specific library member without reference to its spatial coordinates. Examples of such encoding techniques include, but are not limited to, spatial encoding techniques, graphical encoding techniques, including the “tea bag” method, chemical encoding methods, and spectrophotometric encoding methods. One of ordinary skill in the art will realize that the particular encoding method to be used in the present invention must be selected based upon the number of library members desired, and the reaction chemistry employed.

In certain embodiments, molecules of the present invention may be prepared using solid support chemistry known in the art. For example, polypeptides having up to twenty amino acids or more may be generated using standard solid phase technology on commercially available equipment (such as Advanced Chemtech multiple organic synthesizers). In certain embodiments, a starting material or later reactant may be attached to the solid phase, through a linking unit, or directly, and subsequently used in the synthesis of desired molecules. The choice of linkage will depend upon the reactivity of the molecules and the solid support units and the stability of these linkages. Direct attachment to the solid support via a linker molecule may be useful if it is desired not to detach the library member from the solid support. For example, for direct on-bead analysis of biological activity, a stronger interaction between the library member and the solid support may be desirable. Alternatively, the use of a linking reagent may be useful if more facile cleavage of the inventive library members from the solid support is desired.

In regard to automation of the present subject methods, a variety of instrumentation may be used to allow for the facile and efficient preparation of chemical libraries of the present invention, and methods of assaying members of such libraries. In general, automation, as used in reference to the synthesis and preparation of the subject chemical libraries, involves having instrumentation complete one or more of the operative steps that must be repeated a multitude of times because a library instead of a single molecule is being prepared. Examples of automation include, without limitation, having instrumentation complete the addition of reagents, the mixing and reaction of them, filtering of reaction mixtures, washing of solids with solvents, removal and addition of solvents, and the like. Automation may be applied to any steps in a reaction scheme, including those to prepare, purify and assay molecules for use in the compositions of the present invention.

There is a range of automation possible. For example, the synthesis of the subject libraries may be wholly automated or only partially automated. If wholly automated, the subject library may be prepared by the instrumentation without any human intervention after initiating the synthetic process, other than refilling reagent bottles or monitoring or programming the instrumentation as necessary. Although synthesis of a subject library may be wholly automated, it may be necessary for there to be human intervention for purification, identification, or the like of the library members.

In contrast, partial automation of the synthesis of a subject library involves some robotic assistance with the physical steps of the reaction schema that gives rise to the library, such as mixing, stirring, filtering and the like, but still requires some human intervention other than just refilling reagent bottles or monitoring or programming the instrumentation. This type of robotic automation is distinguished from assistance provided by convention organic synthetic and biological techniques because in partial automation, instrumentation still completes one or more of the steps of any schema that is required to be completed a multitude of times because a library of molecules is being prepared.

In certain embodiments, the subject library may be prepared in multiple reaction vessels (e.g., microtitre plates and the like), and the identity of particular members of the library may be determined by the location of each vessel. In other embodiments, the subject library may be synthesized in solution, and by the use of deconvolution techniques, the identity of particular members may be determined.

In one aspect of the invention, the subject screening method may be carried out utilizing immobilized libraries. In certain embodiments, the immobilized library will have the ability to bind to a microorganism as described above. The choice of a suitable support will be routine to the skilled artisan. Important criteria may include that the reactivity of the support not interfere with the reactions required to prepare the library. Insoluble polymeric supports include functionalized polymers based on polystyrene, polystyrene/divinylbenzene copolymers, and the like, including any of the particles described in section 4.3. It will be understood that the polymeric support may be coated, grafted or otherwise bonded to other solid supports.

In another embodiment, the polymeric support may be provided by reversibly soluble polymers. Such polymeric supports include functionalized polymers based on polyvinyl alcohol or polyethylene glycol (PEG). A soluble support may be made insoluble (e.g., may be made to precipitate) by addition of a suitable inert nonsolvent. One advantage of reactions performed using soluble polymeric supports is that reactions in solution may be more rapid, higher yielding, and more complete than reactions that are performed on insoluble polymeric supports.

Once the synthesis of either a desired solution phase or solid support bound template has been completed, the template is then available for further reaction to yield the desired solution phase or solid support bound structure. The use of solid support bound templates enables the use of more rapid split and pool techniques.

Characterization of the library members may be performed using standard analytical techniques, such as mass spectrometry, Nuclear Magnetic Resonance Spectroscopy, including 195Pt and 1H NMR, chromatography (e.g, liquid etc.) and infra-red spectroscopy. One of ordinary skill in the art will realize that the selection of a particular analytical technique will depend upon whether the inventive library members are in the solution phase or on the solid phase. In addition to such characterization, the library member may be synthesized separately to allow for more ready identification.

Any form of the DR6 and/or GFD NAPP polypeptides of the invention may be used to assess the activity of candidate small molecules and other modulators in in vitro assays. The interaction of a DR6 and/or GFD NAPP and a modulator thereof may be determined by any of a variety of techniques known in the art for demonstrating an intermolecular interaction between DR6 and/or GFD NAPP and another molecule, for example, co-purification, co-precipitation, co-immunoprecipitation, radiometric or fluorimetric assays, western immunoblot analyses, affinity capture including affinity techniques such as solid-phase ligand-counterligand sorbent techniques, affinity chromatography and surface affinity plasmon resonance, NMR, and the like (see, e.g., U.S. Pat. No. 5,352,660). Determination of the interaction may employ antibodies, including monoclonal, polyclonal, chimeric and single-chain antibodies, and the like, that specifically bind to DR6 and/or GFD NAPP or the binding agent.

Labeled DR6 and/or GFD NAPP and/or labeled modulator(s) can also be employed to detect the interaction of DR6 and/or GFD NAPP with a modulator. The molecule of interest can be labeled by covalently or non-covalently attaching a suitable reporter molecule or moiety, for example any of various enzymes, fluorescent materials, luminescent materials, and radioactive materials. Examples of suitable enzymes include, but are not limited to, horseradish peroxidase, alkaline phosphatase, beta-galactosidase, and acetylcholinesterase. Examples of suitable fluorescent materials include, but are not limited to, umbelliferone, fluorescein, fluorescein isothiocyanate, rhodamine, dichlorotriazinylamine fluorescein, dansyl chloride, phycoerythrin, Texas Red, AlexaFluor-594, AlexaFluor-488, Oregon Green, BODIPY-FL and Cy-5. Appropriate luminescent materials include, but are not limited to, luminol and suitable radioactive materials include radioactive phosphorus [³²P], iodine [¹²⁵I] or [¹³¹I] or tritium [³H].

DR6 and/or GFD NAPP and the candidate inhibitor may be combined under conditions and for a time sufficient to permit formation of an intermolecular complex between the components. Suitable conditions for formation of such complexes are known in the art and can be readily determined based on teachings provided herein, including solution conditions and methods for detecting the presence of a complex and/or for detecting free substrate in solution. The degree of binding is then measured using an appropriate technique for the reporter molecule being used, all as is well-known in the art.

The extent of association of a detectably candidate modulator(s) in a complex with DR6 and/or NAPP, compared to the fraction of the candidate modulator that is not part of such a complex, may be identified according to a preferred embodiment by detection of a fluorescence energy signal generated by the substrate. Typically, an energy source for detecting a fluorescence energy signal is selected according to criteria with which those having ordinary skill in the art are familiar, depending on the fluorescent reporter moiety with which the substrate is labeled. The test solution, containing (a) DR6 and/or GFD NAPP and (b) the detectably labeled candidate modulator, is exposed to the energy source to generate a fluorescence energy signal, which is detected by any of a variety of well-known instruments and identified according to the particular fluorescence energy signal. In preferred embodiments, the fluorescence energy signal is a fluorescence polarization signal that can be detected using a spectrofluorimeter equipped with polarizing filters. In particularly preferred embodiments the fluorescence polarization assay is performed simultaneously in each of a plurality of reaction chambers that can be read using an LJL CRITERION™ Analyst (LJL Biosystems, Sunnyvale, Calif.) plate reader, for example, to provide a high throughput screen (HTS) having varied reaction components or conditions among the various reaction chambers; Examples of other suitable instruments for obtaining fluorescence polarization readings include the POLARSTAR™ (BMG Lab Technologies, Offenburg, Germany), BEACON™ (Panvera, Inc., Madison, Wis.) and the POLARION™ (Tecan, Inc., Research Triangle Park, N.C.) devices.

Determination of the presence of a complex that has formed between DR6 and/or GFD NAPP and a candidate inhibitor may be performed by a variety of methods, as noted above, including fluorescence energy signal methodology as provided herein and as known in the art. Such methodologies may include, by way of illustration and not limitation FP, FRET, FQ, other fluorimetric assays, co-purification, co-precipitation, co-immunoprecipitation, radiometric, western immunoblot analyses, affinity capture including affinity techniques such as solid-phase ligand-counterligand sorbent techniques, affinity chromatography and surface affinity plasmon resonance, circular dichroism, and the like. For these and other useful affinity techniques, see, for example, Scopes, R. K., Protein Purification: Principles and Practice, 1987, Springer-Verlag, N.Y.; Weir, D. M., Handbook of Experimental Immunology, 1986, Blackwell Scientific, Boston; and Hermanson, G. T. et al., Immobilized Affinity Ligand Techniques, 1992, Academic Press, Inc., California; which are hereby incorporated by reference in their entireties, for details regarding techniques for isolating and characterizing complexes, including affinity techniques. In various embodiments, DR6 and/or GFD NAPP may interact with a binding agent and/or candidate modulator via specific binding if DR6 and/or GFD NAPP binds the binding agent and/or candidate inhibitor with a Kₐ of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹ to 10¹¹ M⁻¹. Affinities of binding partners can be readily calculated from data generated according to the fluorescence energy signal methodologies described above and using conventional data handling techniques, for example, those described by Scatchard et al., Ann. N.Y. Acad. Sci. 51:660 (1949).

For example, in various embodiments where the fluorescence energy signal is a fluorescence polarization signal, fluorescence anisotropy (in polarized light) of the free detectably labeled candidate modulator can be determined in the absence of DR6 and/or GFD NAPP, and fluorescence anisotropy (in polarized light) of the bound substrate can be determined in the presence of a titrated amount of DR6 and/or GFD NAPP. Fluorescence anisotropy in polarized light varies as a function of the amount of rotational motion that the labeled candidate inhibitor and/or binding agent undergoes during the lifetime of the excited state of the fluorophore, such that the anisotropies of free and fully bound candidate modulator can be usefully employed to determine the fraction of candidate modulator and/or binding agent bound to DR6 and/or GFD NAPP in a given set of experimental conditions, for instance, those wherein a candidate agent is present (see, e.g., Lundblad et al., 1996 Molec. Endocrinol. 10:607; Dandliker et al., 1971 Immunochem. 7:799; Collett, E., Polarized Light: Fundamentals and Applications, 1993 Marcel Dekker, New York).

A number of methods for identifying a molecule that modulates the activity of a polypeptide are known in the art. For example, in one such method, a DR6 and/or GFD NAPP protein is contacted with a test compound, and the ability of the DR6 and/or GFD NAPP protein to bind to its counterpart (GFD NAPP or DR6, as the case may be) in the presence of the test compound is determined, wherein a decrease or elimination of the ability of the DR6 and GFD NAPP to bind to each other is indicative that the test compound modulates the activity of the DR6 and/or GFD NAPP.

In certain of the subject assays, to evaluate the results using the subject compositions, comparisons may be made to known molecules, such as one with a known binding affinity for the target. For example, a known molecule and a new molecule of interest may be assayed. The result of the assay for the subject complex will be of a type and of a magnitude that may be compared to result for the known molecule. To the extent that the subject complex exhibits a type of response in the assay that is quantifiably different from that of the known molecule then the result for such complex in the assay would be deemed a positive or negative result. In certain assays, the magnitude of the response may be expressed as a percentage response with the known molecule result, e.g. 100% of the known result if they are the same.

As those skilled in the art will understand, based on the present description, binding assays may be used to detect agents that bind to DR6 and/or GFD NAPP. Cell-free assays may be used to identify molecules that are capable of binding. In a preferred embodiment, cell-free assays for identifying such molecules are comprised essentially of a reaction mixture containing a target and a test molecule or a library of test molecules. A test molecule may be, e.g., a derivative of a known binding partner of the target, e.g., a biologically inactive peptide, or a small molecule. Agents to be tested for their ability to bind may be produced, for example, by bacteria, yeast or other organisms (e.g. natural products), produced chemically (e.g. small molecules, including peptidomimetics), or produced recombinantly. In certain embodiments, the test molecule is selected from the group consisting of lipids, carbohydrates, peptides, peptidomimetics, peptide-nucleic acids (PNAs), proteins, small molecules, natural products, aptamers and oligonucleotides. In other embodiments of the invention, the binding assays are not cell-free. In a preferred embodiment, such assays for identifying molecules that bind a target comprise a reaction mixture containing a target microorganism and a test molecule or a library of test molecules.

In many candidate screening programs which test libraries of molecules and natural extracts, high throughput assays are desirable in order to maximize the number of molecules surveyed in a given period of time. Assays of the present invention which are performed in cell-free systems, such as may be derived with purified or semi-purified proteins or with lysates, are often preferred as “primary” screens in that they may be generated to permit rapid development and relatively easy detection of binding between a target and a test molecule. Moreover, the effects of cellular toxicity and/or bioavailability of the test molecule may be generally ignored in the in vitro system, the assay instead being focused primarily on the ability of the molecule to bind the target. Accordingly, potential binding molecules may be detected in a cell-free assay generated by constitution of functional interactions of interest in a cell lysate. In an alternate format, the assay may be derived as a reconstituted protein mixture which, as described below, offers a number of benefits over lysate-based assays.

In one aspect, the present invention provides assays that may be used to screen for molecules that bind DR6 and/or GFD NAPP druggable regions and thus prevent or inhibit the binding of DR6 to GFD NAPP. In an exemplary binding assay, the molecule of interest is contacted with a mixture generated from target cell surface polypeptides. Detection and quantification of expected binding to a target polypeptide provides a means for determining the molecule's efficacy at binding the target. The efficacy of the molecule may be assessed by generating dose response curves from data obtained using various concentrations of the test molecule. Moreover, a control assay may also be performed to provide a baseline for comparison. In the control assay, the formation of complexes is quantitated in the absence of the test molecule.

Complex formation between a molecule and a DR6 and/or GFD NAPP protein or microorganism containing a DR6 and/or GFD NAPP protein may be detected by a variety of techniques, many of which are described above. For instance, modulation in the formation of complexes may be quantitated using, for example, detectably labeled proteins (e.g. radiolabeled, fluorescently labeled, or enzymatically labeled), by immunoassay, or by chromatographic detection.

Accordingly, one exemplary screening assay of the present invention includes the steps of contacting a DR6 and/or GFD NAPP polypeptide of the invention with a test molecule or library of test molecules and detecting the formation of complexes. For detection purposes, for example, the molecule may be labeled with a specific marker and the test molecule or library of test molecules labeled with a different marker. Interaction of a test molecule with a polypeptide or fragment thereof may then be detected by determining the level of the two labels after an incubation step and a washing step. The presence of two labels after the washing step is indicative of an interaction. Such an assay may also be modified to work with a whole target cell.

An interaction between a DR6 and/or GFD NAPP protein and a candidate molecule may also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test molecules may be immobilized on a sensor surface, e.g., which forms one wall of a micro-flow cell. A solution containing the target is then flowed continuously over the sensor surface. A change in the resonance angle as shown on a signal recording, indicates that an interaction has occurred. This technique is further described, e.g., in BIAtechnology Handbook by Pharmacia.

For example, it may be desirable to immobilize the target to facilitate separation of complexes from uncomplexed forms, as well as to accommodate automation of the assay. Binding of polypeptide to a test molecule may be accomplished in any vessel suitable for containing the reactants. Examples include microtitre plates, test tubes, and micro-centrifuge tubes. In one embodiment, a fusion protein may be provided which adds a domain that allows the target to be bound to a matrix. For example, glutathione-S-transferase/polypeptide (GST/polypeptide) fusion proteins may be adsorbed onto glutathione sepharose beads (Sigma Chemical, St. Louis, Mo.) or glutathione derivatized microtitre plates, which are then combined with a labeled test molecule (e.g., S³⁵ labeled, P³³ labeled, and the like, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintillant), or in the supernatant after the complexes are subsequently dissociated. Alternatively, the complexes may be dissociated from the matrix, separated by SDS-PAGE, and the level of polypeptide or binding partner found in the bead fraction quantitated from the gel using standard electrophoretic techniques such as described in the appended examples. The above techniques could also be modified in which the test molecule is immobilized, and the labeled target is incubated with the immobilized test molecules. In one embodiment of the invention, the test molecules are immobilized, optionally via a linker, to a particle of the invention, e.g. to create the ultimate composition.

Other techniques for immobilizing targets or molecules on matrices may be used in the subject assays. For instance, a target or molecule may be immobilized utilizing conjugation of biotin and streptavidin. For instance, biotinylated polypeptide molecules may be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a target or molecule may be derivatized to the wells of the plate, and the target or molecule trapped in the wells by antibody conjugation. As above, preparations of test molecules are incubated in the polypeptide presenting wells of the plate, and the amount of complex trapped in the well may be quantitated. Exemplary methods for detecting such complexes, in addition to those described above for the GST-immobilized complexes, include immunodetection of complexes using antibodies reactive with the complex, or which are reactive with one of the complex components; as well as enzyme-linked assays which rely on detecting an enzymatic activity associated with a target or molecule, either intrinsic or extrinsic activity. In an instance of the latter, the enzyme may be chemically conjugated or provided as a fusion protein with the target or molecule. To illustrate, a target polypeptide may be chemically cross-linked or genetically fused with horseradish peroxidase, and the amount of polypeptide trapped in a complex with a molecule may be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase may be provided, and complex formation quantitated by detecting the GST activity using 1-chloro-2,4-dinitrobenzene (e.g. Habig et al (1974) J Biol Chem 249:7130).

For processes that rely on immunodetection for quantitating one of the components trapped in a complex, antibodies against a component, such as anti-polypeptide antibodies, may be used. Alternatively, the component to be detected in the complex may be “epitope tagged” in the form of a fusion protein which includes, in addition to the polypeptide sequence, a second polypeptide for which antibodies are readily available (e.g. from commercial sources). For instance, the GST fusion proteins described above may also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

In certain in vitro embodiments of the present assay, the solution containing the target comprises a reconstituted protein mixture of at least semi-purified proteins. By semi-purified, it is meant that the components utilized in the reconstituted mixture have been previously separated from other cellular or viral proteins. For instance, in contrast to cell lysates, a target protein is present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which interferes with or otherwise alters the ability to measure binding activity. In one embodiment, the use of reconstituted protein mixtures allows more careful control of the target:molecule interaction conditions.

All of the screening methods may be accomplished by using a variety of assay formats. In light of the present disclosure, those not expressly described herein will nevertheless be known and comprehended by one of ordinary skill in the art. Assay formats which approximate such conditions as formation of protein complexes or protein-nucleic acid complexes, and enzymatic activity may be generated in many different forms, as those skilled in the art will appreciate based on the present description and include but are not limited to assays based on cell-free systems, e.g. purified proteins or cell lysates, as well as cell-based assays which utilize intact cells. Assaying binding resulting from a given target:molecule interaction may be accomplished in any vessel suitable for containing the reactants. Examples include microtitre plates, test tubes, and micro-centrifuge tubes. Any of the assays may be provided in kit format and may be automated. Many of the following particularized assays rely on general principles, such as blockage or prevention of fusion, that may apply to other particular assays.

In any of the assays described herein, a test cell may express the DR6 and/or GFD NAPP protein naturally or following introduction of a recombinant DNA molecule encoding the protein. Transfection and transformation protocols are well known in the art and include Ca₂PO₄-mediated transfection, electroporation, infection with a viral vector, DEAE-dextran mediated transfection, and the like. As an alternative to the proteins described above, chimeric DR6 and/or GFD NAPP proteins (ie., fusion of DR6 and/or GFD NAPP protein with another protein or protein fragment), or protein sequences engineered to lack a leader sequence may be employed. In a similar fashion, a fusion may be constructed to direct secretion, export, or cytosolic retention. Any and all of these sequences may be employed in a fusion construct with DR6 and/or GFD NAPP to assist in assaying modulators. The host cell can also express DR6 and/or GFD NAPP as a result of being diseased, infected with a virus, and the like. Secreted proteins that are exported by virtue of a leader sequence are well known and include, human chorionic gonadatropin (hCG.alpha.), growth hormone, hepatocyte growth factor, transferrin, nerve growth factor, vascular endothelial growth factor, ovalbumin, and insulin-like growth factor. Similarly, cytosolic proteins are well known and include, neomycin phosphotransferase, beta-galactosidase, actin and other cytoskeletal proteins, and enzymes, such as protein kinase A or C. The most useful cytosolic or secreted proteins are those that are readily measured in a convenient assay, such as ELISA. The three proteins (leaderless, secreted, and cytosolic) may be co-expressed naturally, by co-transfection in the test cells, or transfected separately into separate host cells. Furthermore, for the assays described herein, cells may be stably transformed or express the protein transiently.

Immunoprecipitation is one such assay that may be employed to determine inhibition. Briefly, cells expressing DR6 and/or GFD NAPP naturally or from an introduced vector construct are labeled with ³⁵S-methionine and/or ³⁵S-cysteine for a brief period of time, typically 15 minutes or longer, in methionine- and/or cysteine-free cell culture medium. Following pulse labeling, cells are washed with medium supplemented with excess unlabeled methionine and cysteine plus heparin if the leaderless protein is heparin binding. Cells are then cultured in the same chase medium for various periods of time. Candidate inhibitors or enhancers are added to cultures at various concentration. Culture supernatant is collected and clarified. Supernatants are incubated with anti-DR6 and/or anti-GFD NAPP immune serum or a monoclonal antibody, or with anti-tag antibody if a peptide tag is present, followed by a developing reagent such as Staphylococcus aureus Cowan strain I, protein A-Sepharose.R™, or Gamma-bind™G-Sepharose.R™. Immune complexes are pelleted by centrifugation, washed in a buffer containing 1% NP-40 and 0.5% deoxycholate, EGTA, PMSF, aprotinin, leupeptin, and pepstatin. Precipitates are then washed in a buffer containing sodium phosphate pH 7.2, deoxycholate, NP-40, and SDS. Immune complexes are eluted into an SDS gel sample buffer and separated by SDS-PAGE. The gel is processed for fluorography, dried, and exposed to x-ray film.

Alternatively, ELISA may be used to detect and quantify the amount of DR6 and/or GFD NAPP in cell supernatants. ELISA is used for the detection in high throughput screening. Briefly, 96-well plates are coated with an anti-DR6 and/or GFD NAPP antibody or anti-tag antibody, washed, and blocked with 2% BSA. Cell supernatant is then added to the wells. Following incubation and washing, a second antibody (e.g., to DR6 and/or GFD NAPP) is added. The second antibody may be coupled to a label or detecting reagent, such as an enzyme or to biotin. Following further incubation, a developing reagent is added and the amount of DR6 and/or GFD NAPP determined using an ELISA plate reader. The developing reagent is a substrate for the enzyme coupled to the second antibody (typically an anti-isotype antibody) or for the enzyme coupled to streptavidin. Suitable enzymes are well known in the art and include horseradish peroxidase, which acts upon a substrate (e.g., ABTS) resulting in a colorimetric reaction. It is recognized that rather than using a second antibody coupled to an enzyme, the anti-DR6 and/or anti-GFD NAPP antibody may be directly coupled to the horseradish peroxidase, or other equivalent detection reagent. If desired, cell supernatants may be concentrated to raise the detection level. Further, detection methods, such as ELISA and the like may be employed to monitor intracellular as well as extracellular levels of DR6 and/or GFD NAPP. When intracellular levels are desired, a cell lysate is used. When extracellular levels are desired, media can be screened.

ELISA may also be readily adapted for screening multiple candidate modulators or with high throughput. Briefly, such an assay is conveniently cell based and performed in 96-well plates. Test cells that naturally or stably express DR6 and/or GFD NAPP are plated at a level sufficient for expressed product detection, such as, about 20,000 cells/well. However, if the cells do not naturally express the protein, transient expression is achieved, such as by electroporation or Ca₂PO₄-mediated transfection. For electroporation, 100 .mu.l of a mixture of cells (e.g., 150,000 cells/ml) and vector DNA (5 .mu.g/ml) is dispensed into individual wells of a 96-well plate. The cells are electroporated using an apparatus with a 96-well electrode (e.g., ECM 600 Electroporation System, BTX, Genetronics, Inc.). Optimal conditions for electroporation are experimentally determined for the particular host cell type. Voltage, resistance, and pulse length are the typical parameters varied. Guidelines for optimizing electroporation may be obtained from manufacturers or found in protocol manuals, such as Current Protocols in Molecular Biology (Ausubel et al. (ed.), Wiley Interscience, 1987). Cells are diluted with an equal volume of medium and incubated for 48 hours. Electroporation may be performed on various cell types, including mammalian cells, yeast cells, bacteria, and the like. Following incubation, medium with or without inhibitor is added and cells are further incubated for 1-2 days. At this time, culture medium is harvested and the protein is assayed by any of the assays herein. Preferably, ELISA is employed to detect the protein.

It should be noted that as used herein the terms “first,” “second,” and the like, as well as “primary,” “secondary,” and the like, do not denote any amount, order, or importance, but rather are used to distinguish one element from another, and the terms “a” and “an” do not denote a limitation of quantity, but rather denote the presence of at least one of the referenced item. As used herein the term “about”, when used in conjunction with a number in a numerical range, is defined being as within one standard deviation of the number “about” modifies. The suffix “(s)” as used herein is intended to include both the singular and the plural of the term that it modifies, thereby including one or more of that term (e.g., the bearings(s) includes one or more bearings). The endpoints of all ranges directed to the same component or property are inclusive and independently combinable (e.g., ranges of “up to about 5°, or, more specifically, about 0.5° to about 3°” is inclusive of the endpoints and all intermediate values of the ranges of “about 0.5° to about 5°,” etc.).

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims. It is, of course, contemplated that alternative methods, which are well known in the art, may be employed.

We claim:

1. A polypeptide comprising about 75% homology to residues 96-167 of death cell receptor six (DR6) as identified in SEQ ID NO: 2, and wherein said polypeptide includes a first Cysteine Rich Domain (CRD) with at least about 75% homology to amino acid residues 96 to 131 of DR6 as identified in SEQ ID NO: 2, and a second CRD with at least about 75% homology to amino acid residues 133 to 167 of DR6 as identified in SEQ ID NO: 2 and wherein residue 98 is Arginine, residue 104 is Glutamic acid, residue 131 is Cysteine, residue 132 is Threonine, residue 139 is Glutamine, residue 163 is Threonine, and residue 167 is Arginine.

2. The polypeptide according to claim 1 comprising about 90% homology to residues 96 to 167 of death cell receptor six (DR6) as identified in SEQ ID NO: 2.

3. The polypeptide according to claim 1 comprising about 100% homology to residues 96 to 167 of death cell receptor six (DR6) as identified in SEQ ID NO: 2.

4. A polypeptide consisting of about 30% homology to residues 96-167 of death cell receptor six (DR6) as identified in SEQ ID NO: 2, and where said polypeptide includes a first Cysteine Rich Domains (CRD) with at least about 30% homology to amino acid residues 96 to 131 of DR6 as identified in SEQ ID NO: 2, and a second CRD with at least about 30% homology to amino acid residues 133 to 167 of DR6 as identified in SEQ ID NO: 2 and wherein residue 98 is Arginine, residue 104 is Glutamic acid, residue 131 is Cysteine, residue 132 is Threonine, residue 139 is Glutamine, residue 163 is Threonine, and residue 167 is Arginine.

5. The polypeptide according to claim 4 which further comprises a disulfide bridge between residues 113 and 131 and a disulfide bridge between residues 133 and 144.

6. The polypeptide according to claim 4 consisting of about 40% homology to residues 96 to 167 of death cell receptor six (DR6) as identified in SEQ ID NO: 2.

7. The polypeptide according to claim 4 consisting of about 50% homology to residues 96 to 167 of death cell receptor six (DR6) as identified in SEQ ID NO: 2.

8. The polypeptide according to claim 4 consisting of about 75% homology to residues 96 to 167 of death cell receptor six (DR6) as identified in SEQ ID NO: 2.

9. The polypeptide according to claim 4 consisting of about 90% homology to residues 96 to 167 of death cell receptor six (DR6) as identified in SEQ ID NO: 2.

10. The polypeptide according to claim 4 consisting of about 100% homology to residues 96 to 167 of death cell receptor six (DR6) as identified in SEQ ID NO: 2.


SEQUENCE LISTING (4 sequences)

SEQ ID NO: 1 - protein, 86 aa, Homo sapiens
  SITE (1)..(1): Cysteine
CGRLNMHMNVQNGKWDSDPSGTKTCIDTKEGILQYCQEVYPELQITNVVEANQPVTIQNWCKRGRKQCKTHPHFVIPYRCLVGEFV

SEQ ID NO: 2 - protein, 72 aa, Homo sapiens
  SITE (1)..(1): Phenylalanine
FTRHENGIEKCHDCSQPCPWPMIEKLPCAALTDRECTCPPGMFQSNATCAPHTVCPVGWGVRKKGTETEDVR

SEQ ID NO: 3 - protein, 94 aa, Homo sapiens
AVAEPQIAMFCGKLNMHVNIQTGKWEPDPTGTKSCFETKEEVLQYCQEMYPELQITNVMEANQRVSIDNWCRRDKKQCKSRFVTPFKCLVGEFV

SEQ ID NO: 4 - protein, 127 aa, Homo sapiens
CKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVC